SEQUENCE LISTING (7 sequences)

SEQ ID NO: 1 - protein, 471 aa, Defluviimonas alba
MTKLIKNGTIVTATDIYEADLLIQDGKIAVIGRNLDESGAEVIDATGCYVFPGGIDPHTHLDMPFGGTVTKDDFESGTIAAAFGGTTTIIDFCLTNKGEPLKKAIETWHNKATGKAVIDYGFHLMISEITDDVLEELPKVIEEEGITSFKVFMAYKDVFQADDGTLYRTLVAAKELGALVMVHAENGDVIDYLTKKALEDGHTDPIYHALTRPPELEGEATGRACQLTELAGSQLYVVHVSCAQAVEKIAEARNKGLNVWGETCPQYLVLDQSYLEKPNFEGAKYVWSPPLREKWHQEVLWNALKNGQLQTLGSDQCSFDFKGQKELGRGDFTKIPNGGPIIEDRVSILFSEGVKKGRITLNQFVDIVSTRIAKLFGLFPKKGTIAVGADADLVIFDPTVERVISAETHHMAVDYNPFEGMKVTGEPVSVLCRGEFVVRDKQFVGKPGYGQYVKRAKYGALMADQDVVKMS

SEQ ID NO: 2 - protein, 395 aa, Cloacibacillus sp.
MNCVNDILRSIGKAGRNEDGSYTRACYSAEYFAAVDITEKLMREYGMETSRDAAGNLHGVLPGTEPGLKSIIIGSHLDTVPEGGLFDGAYGVAGGLEVVRRLKEEGRRPRHTIELYGFNAEESSPLGGTFGSRAVTGLVSPEQPGLAEALKSYGHTVEEIMGCRRDFSDAKCYLELHIEQGDYLFSEGQKIGVVSGIVGVIRYKVTALGHSNHAGTTMMKNRRDAMVAMARLITEADRRCRAIDDRLVLTVGTIKCWPGSENVIPGKVECSFEMRHMDKAKTDELIREIREIAENIATVEFEIVNMIDKGAVSCDAHLMDVICEAAEEAGESHVVMPSGAGHDANPMAHRVPIGMIFVPSKDGMSHCPEEWTDSEETAAGAEVLYRTVLALDAED

SEQ ID NO: 3 - protein, 425 aa, Burkholderia lata
MNPTDFPFPPLNAERLNARVEQLARFTRPDVPWTRRAFSPLFTEARAWLAAQFAEAGLAVSMDAGGNLIGRREGSGRCTKPLVTGSHCDTVVGGGRFDGIIGVLAGIEVAHTLNEQGIVLDHPFEVIDFLSEEPSDYGISCVGSRALSGVLDAGMLRATNAEGETLAEALRRIGGNPDALREPLRAPGSTAAFVELHIEQGPVLETRGLPIGVVTNIVGIRRVLITVTGQPDHAGTTPMDIRRDALVGAAHLIEAAHARASALSGNPHYVVATIGRIAMTPNVPNAVPGQVELMLEVRSDSDAVLDAFPEALLAGAAARLDALRLSARAEHVSRARPTDCQPLVMDAVEQAATQLGYPSMRLPSGAGHDAVYVAPTGPIGMIFIPCLGGRSHCPEEWIEPQQLLDGTRVLYQTLVALDRSLAGAA

SEQ ID NO: 4 - protein, 414 aa, Chloroflexus sp.
MTDAARLERRIHELAQIGRTDDPAREIYATAVSRLGLSAEEQRARDLVTSWCAPHGATARRDPAANLYLRFPGADPHAPVVLVGSHLDSVPMGGRFDGALGVCCAVEAVVSLLESGARFARPVEVVGWADEEGARFGYGLFGSAAAFGRLRVDPERVRDKGGTSIAEALRALGESGDLAGAMRDPKGIRAYLELHIEQGPRLERAGAPLGVVSDIVGIFHGLVMVRGEQNHAGATVMGERHDALVAASHMIIALERIASSVPDAVATVGEITVKPGAKNVIPGECTFSLDIRAPKQESIDLVLERFKAEANEIFRKSLREWGLRPLQSVAVTPLDEDLRDLLWKSAMSVGVNAPTLVSGAGHDAQNPSLAGVPTGMIFVRSTGGSHTPTEFAATADAALGAKALEIAIRELATA

SEQ ID NO: 5 - protein, 240 aa, Rhizobium radiobacter
MHIRLINPNSTASMTAQALDSALRVKQKDTHVSAANPVDTPVSIEGQADEAMAVPGLLAEIRKGEGHGVDAYVIACFDDPGLHAAREVARGPVIGICQAAVQVAMTISRRFSIITTLPRSIPIIEDLVEDYGAQRYCRKVRAIDLPVLGLEEDPEVAEALLRREIEAAKREDAAEAIILGCAGMSSLCDRLRDATGVPVIDGVTAAIKLAEALVGAGYTTSKVNAYDYPRVKGPALVACA

SEQ ID NO: 6 - protein, 223 aa, Yoonia sediminilitoris
MSALIIINPNSSQTVTDGIDAAVAPLRSFGTPIRCLTLAEGPPGIESQKQADLTVAPMLKLAAEQADAAGYVIACFGDPGLHALRDQTHLPVVGIQEAAVMTALTLGQRFGVIAIMPGSIPRHLRAFGAMSVLDRLAGDRALGLGVADLADPDRSLAAMIATGKRLRDEDGAHVLIMGCAGMAHYRPTLETETGLPVVEPCQAATAMVLGHIALGQSHRRDQN

SEQ ID NO: 7 - protein, 680 aa, Rhizobium radiobacter
MDIIIKNGTIVTADGISPADLGIKDGKIAQIGGTFGPAGRTIDASGRYVFPGGIDVHTHVETVSFNTQSADTFATATVAAACGGTTTIVDFCQQDRGHSLREAVAKWDGMAGGKSAIDYGYHIIVLDPTDSVIEELEVLPDLGITSFKVFMAYRGMNMIDDVTLLRTLDKAAKTGSLVMVHAENGDAADYLRDKFVADGKTAPIYHALSRPPRVEAEATARALALAEIVNAPIYIVHLTCEESFDELMRAKARGVHALAETCTQYLYLTKDDLERPDFEGAKYVFTPPPRTKKDQEILWNALRNGVLETVSSDHCSWLFEGHKDRGRNDFRAIPNGAPGVEERLMMVYQGVNEGRISLTQFVELVATRPAKVFGMFPEKGTVAVGSDADIVLWDPEAEMVIEQSAMHNAMDYSSYEGHKIKGVPKTVLLRGKVIVDEGTYVGAPTDGQFRKRRKYKQMSALIIINPNSSQTVTDGIDAAVAPLRSFGTPIRCLTLAEGPPGIESQKQADLTVAPMLKLAAEQADAAGYVIACFGDPGLHALRDQTHLPVVGIQEAAVMTALTLGQRFGVIAIMPGSIPRHLRAFGAMSVLDRLAGDRALGLGVADLADPDRSLAAMIATGKRLRDEDGAHVLIMGCAGMAHYRPTLETETGLPVVEPCQAATAMVLGHIALGQSHRRDQN

(19) United States
(12) Patent Application Publication
(10) Pub. No.: US 2025/0034605 A1
(43) Pub. Date: Jan. 30, 2025

(54) ENZYMATIC DECARBAMOYLATION OF GLUFOSINATE DERIVATIVES

(71) Applicant: BASF SE, Ludwigshafen am Rhein (DE)

(72) Inventors: Gunther Zimmermann, Ludwigshafen (DE); Moritz Stefan Pott, Ludwigshafen (DE); Michael Breuer, Ludwigshafen (DE); Klaus Ditrich, Goennheim (DE); Stefan Seemayer, Ludwigshafen (DE)

(21) Appl. No.: 18/717,729

(22) PCT Filed: Dec. 12, 2022
(86) PCT No.: PCT/EP2022/085314

§ 371 Date: Jun. 7, 2024

(30) Foreign Application Priority Data
Dec. 10, 2021 (EP) 21213752.5

(51) Int. Cl. C12P 9/00; A01N 57/20; C12N 9/80; C12N 9/86; C12N 9/90; C12P 41/00

(52) U.S. Cl. CPC C12P 9/00; A01N 57/20; C12N 9/80; C12N 9/86; C12N 9/90; C12P 41/009; C12Y 305/01087; C12Y 305/02002; C12Y 501/0101

(57) ABSTRACT

The present invention relates to a method of manufacturing glufosinate, comprising the step of enzymatically cleaving off a carbamoyl moiety of a carbamoyl amino acid compound.

The present application is directed to a method of manufacturing glufosinate, comprising the step of enzymatically cleaving off the carbamoyl moiety of a N-carbamoyl amino acid compound.

The herbicide glufosinate is a non-selective, foliarly-applied herbicide considered to be one of the safest herbicides from a toxicological or environmental standpoint. Current commercial chemical synthesis methods for glufosinate yield a racemic mixture of L- and D-glufosinate (Duke et al. 2010 Toxins 2:1943-1962).

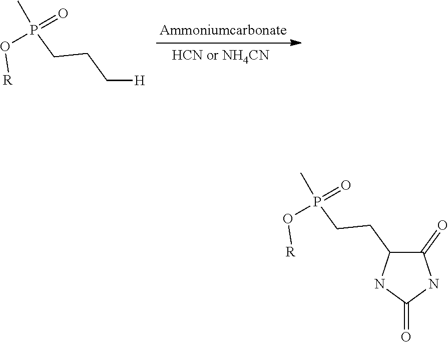

Scheme 1. Syntheses of hydantoin via the respective aldehyde (wherein R is e.g. H or alkyl). It is known that hydantoins can be intermediates in the synthesis of racemic Glufosinate. They may be accessed from the respective aldehydes (Scheme 1) by the Bucherer-Bergs Reaction. A further synthesis route is e.g. described in CN 111662325.

CN113045604 discloses a method of synthesizing glufosinate starting from a hydantoin. The reaction needs to be performed under high pressure and at temperatures between 13° and 180° C. Hence, the reaction conditions are rather harsh. However, using an autoclave and/or temperatures above 120° C. on an industrial scale is also connected with a safety risk for the coworkers. CN 111662325 also describes the hydrolysis of an hydantoin to obtain Glufosinate, however the reaction requires strong acids or bases and refluxing conditions in water.

It is known that L-glufosinate (also known as phosphinothricin or (S)-2-amino-4-(hydroxy(methyl) phosphonoyl)butanoic acid) is more potent than D-glufosinate (Ruhland et al. (2002) Environ. Biosafety Res. 1:29-37). Therefore, methods to produce the more active L-glufosinate form in excess are of further interest.

Against the above background, it has been an object of the present invention to provide a mild method of manufacturing glufosinate.

It has further been an object of the present invention to provide a safe method of manufacturing glufosinate.

It has further been an object of the present invention to provide a mild method of manufacturing L-glufosinate in an enantiomeric excess.

It has further been an object of the present invention to provide a composition comprising L-glufosinate.

It has further been an object of the present invention to provide a method for selectively controlling weeds using the composition as obtained according to the inventive method of manufacturing.

It has surprisingly been found by the inventors of the present invention that at least one of the above objects can be obtained by the herein described N-carbamoyl amino acid-based process. It has further been found by the inventors of the present invention that the claimed method provides a composition comprising glufosinate in a sufficient amount for using as herbicide.

In a first aspect, the present invention therefore relates to a method of manufacturing glufosinate, its alkyl ester or the salts thereof having the formula (3)

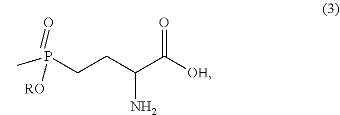

wherein R is H or C1-C8alkyl, comprising the step of enzymatically cleaving off the carbamoyl moiety of a N-carbamoyl amino acid having the formula (2)

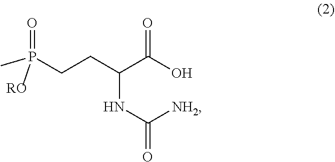

wherein R is H or C1-C8alkyl.

In the following, preferred embodiments of the components of the method of manufacturing, the composition, and the method of selectively controlling weeds are described in further detail. It is to be understood that each preferred embodiment is relevant on its own as well as in combination with other preferred embodiments.

In a preferred embodiment A1 of the first aspect, the cleaving step provides glufosinate, its alkyl ester or the salts thereof having the formula (3)

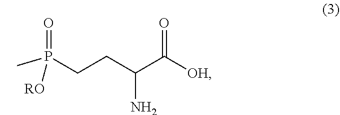

wherein R is H or C1-C8alkyl, preferably H or C1-C6alkyl, more preferably H or C2-C4alkyl, even more preferably H, ethyl, or butyl, and in particular H.

In a preferred embodiment A2 of the first aspect, the cleaving step provides glufosinate, its alkyl ester or the salts thereof having the formula (3) in form of a racemic mixture or in form of an enantiomeric excess of L-glufosinate, its alkyl ester or the salts thereof having the formula (3a)

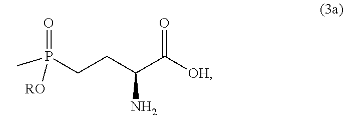

wherein R is H or C1-C8alkyl, preferably H or C1-C6alkyl, more preferably H or C2-C4alkyl, even more preferably H, ethyl, or butyl, and in particular H; preferably in form of an enantiomeric excess of L-glufosinate, its alkyl ester or the salts thereof having the formula (3a).

In a preferred embodiment A3 of the first aspect, at least 50%, preferably at least 60%, and in particular at least 80%, of the N-carbamoyl amino acid having the formula (2) is converted to L-glufosinate, its alkyl ester or the salts thereof having the formula (3a), wherein formula (3a) is as defined in embodiment A2.

In a preferred embodiment A4 of the first aspect, the cleaving is performed by an N-Carbamoyl amino acid hydrolase enzyme, preferably an L-N-Carbamoyl amino acid hydrolase enzyme.

In a preferred embodiment A5 of the first aspect, the cleaving is performed by an N-Carbamoyl amino acid hydrolase enzyme selected from the group consisting of Uniprot ID: A0A1Y4GC62_9BACT (SEQ ID NO:2), Uniprot ID: A0A6P2ISL4_BURL3 (SEQ ID NO:3), and mixtures thereof.

In a preferred embodiment A6 of the first aspect, R in formulae (2) and (3) is H or C1-C6alkyl, preferably H or C2-C4alkyl, more preferably H, ethyl, or butyl, and in particular H.

In a preferred embodiment A7 of the first aspect, the cleaving step is performed at a pH of 6 to 11, preferably of 6.5 to 10, more preferably of 7 to 9.5, and in particular of 7.5 to 9 and/or at a temperature of 20 to 50° C., preferably of 25 to 45° C., more preferably of 30 to 42° C., and in particular of 32 to 40° C.

In a preferred embodiment A8 of the first aspect, R in formulae (2) and (3) is C1-C8alkyl, preferably C1-C6alkyl, more preferably C2-C4alkyl, even more preferably ethyl or butyl, and in particular ethyl, and the method further comprises the step of
c) deprotecting under acidic conditions, preferably using hydrochloric acid or sulfuric acid.

In a preferred embodiment A9 of the first aspect, the method further comprises the addition of an N-Carbamoyl amino acid racemase enzyme.

In a preferred embodiment A10 of the first aspect, the N-carbamoyl amino acid having the formula (2) is provided by a preceding hydrolysing step comprising hydrolysing a hydantoin having the formula (1)

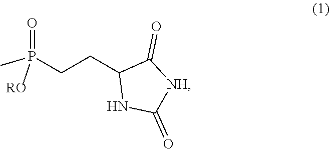

wherein R is H or C1-C8alkyl, to form the N-carbamoyl amino acid having the formula (2), which preferably is performed under enzymatic conditions

In a preferred embodiment A11 of the first aspect, the N-carbamoyl amino acid having the formula (2) is provided by a preceding hydrolysing step, wherein hydrolysing the hydantoin having the formula (1) is performed by a Hydantoinase enzyme.

In a preferred embodiment A12 of the first aspect, the method further comprises the addition of an Hydantoin Racemase enzyme.

In a preferred embodiment A13 of the first aspect, the hydrolysing step and the cleaving step are performed in a single container, preferably wherein all reagents are substantially added at the start of the reaction or wherein the reagents for the hydrolysing step and the reagents for the cleaving step are added to the single container at different times.

In a second aspect, the present invention relates to composition comprising a N-carbamoyl amino acid having the formula (2a)

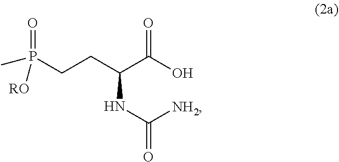

wherein R is H or C1-C8alkyl, and L-glufosinate or the salts thereof.

In a third aspect, the present invention relates to a method for selectively controlling weeds in an area, preferably containing a crop of planted seeds or crops that are resistant to glufosinate, comprising:
applying an effective amount of a composition comprising L-glufosinate or the salts thereof at an enantiomeric proportion of at least 80% over D-glufosinate or the salts thereof and more than 0.01 wt.-% to less than 10 wt.-%, based on the total amount of the composition, of a N-carbamoyl amino acid having the formula (2)

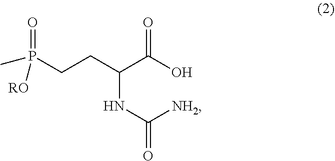

wherein R is H or C1-C8alkyl, to the area.

### DETAILED DESCRIPTION

Before describing in detail exemplary embodiments of the present invention, definitions important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably +10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The term “wt.-%” as used throughout herein stands for “percent by weight”.

The term “alkyl” as used herein denotes in each case a straight-chain or branched alkyl group having usually from 1 to 20 carbon atoms, preferably from 1 to 8 carbon atoms, frequently from 1 to 6 carbon atoms, more preferably 1 to 4 carbon atoms, e.g. 2 or 4 carbon atoms. Examples of alkyl groups are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, and n-hexyl.

Depending on the substitution pattern, the compounds according to the invention may have one or more stereocenters. Unless explicitly indicated otherwise (e.g. via a chemical formula) the invention preferably encompasses all stereoisomers, i.e. pure enantiomers, pure diastereomers, of the compounds according to the invention, and their mixtures, including racemic mixtures.

Preferred embodiments regarding the method of manufacturing glufosinate, its alkyl ester or the salts thereof having the formula (3), the composition comprising a N-carbamoyl amino acid having the formula (2a) and L-glufosinate or the salts thereof, and the method for selectively controlling weeds are described in detail hereinafter. It is to be understood that the preferred embodiments of the invention are preferred alone or in combination with each other.

As indicated above, the present invention relates in one aspect to a method of manufacturing glufosinate, its alkyl ester or the salts thereof having the formula (3)

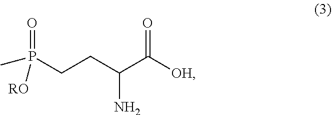

wherein R is H or C1-C8alkyl, comprising the step of enzymatically cleaving off the carbamoyl moiety of a N-carbamoyl amino acid having the formula (2)

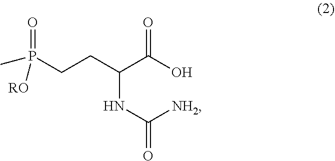

wherein R is H or C1-C8alkyl.

It is to be understood that the glufosinate, its alkyl ester or the salts thereof having the formula (3)

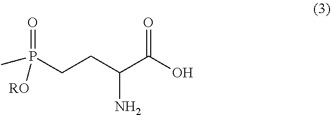

encompasses all stereoisomers, suitable salts of the respective glufosinate or its alkyl ester. Further, the respective zwitterions are encompassed by the formula (3). Suitable salts are exemplarily hydrochloric acid salt, ammonium salts, and isopropylammonium salts. In this connection, the compound of formula (3) in particular encompasses two stereocenters, wherein one stereocenter is located at the phosphor atom and one stereocenter is located at the alpha carbon atom. The compound of formula (3) in particular encompasses all stereoisomers derived from the stereocenter at the phosphor atom.

In a preferred embodiment, the cleaving step provides glufosinate, its alkyl ester or the salts thereof having the formula (3)

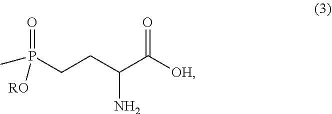

wherein R is H or C1-C8alkyl, preferably H or C1-C6alkyl, more preferably H or C2-C4alkyl, even more preferably H, ethyl, or butyl, and in particular H.

In a preferred embodiment of the present invention, the cleaving step provides glufosinate, its alkyl ester or the salts thereof having the formula (3) in form of a racemic mixture.

In another preferred embodiment of the present invention, the cleaving step provides glufosinate, its alkyl ester or the salts thereof having the formula (3) in form of an enantiomeric excess of L-glufosinate, its alkyl ester or the salts thereof having the formula (3a)

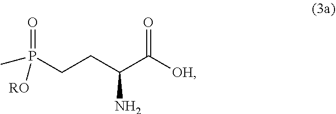

wherein R is H or C1-C8alkyl, preferably H or C1-C6alkyl, more preferably H or C2-C4alkyl, even more preferably H, ethyl, or butyl, and in particular H.

In another preferred embodiment of the present invention, the cleaving step provides glufosinate, its alkyl ester or the salts thereof having the formula (3) in form of an enantiomeric excess of D-glufosinate, its alkyl ester or the salts thereof having the formula (3b)

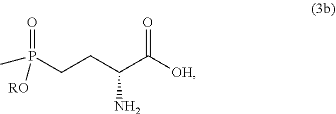

wherein R is H or C1-C8alkyl, preferably H or C1-C6alkyl, more preferably H or C2-C4alkyl, even more preferably H, ethyl or butyl, and in particular H.

In a preferred embodiment of the present invention, at least 50%, preferably at least 60%, more preferably at least 80%, even more preferably at least 90%, and in particular at least 95%, of the N-carbamoyl amino acid having the formula (2) is converted to L-glufosinate, its alkyl ester or the salts thereof having the formula (3a), wherein R of formula (3a) is H or C1-C8alkyl, preferably H or C1-C6alkyl, more preferably H or C2-C4alkyl, even more preferably H, ethyl, or butyl, and in particular H.

In a preferred embodiment of the present invention, 50 to 99.9%, preferably 60 to 99.8%, more preferably 80 to 99.5, even more preferably 90 to 99.2%, and in particular 95 to 99%, of the N-carbamoyl amino acid having the formula (2) is converted to L-glufosinate, its alkyl ester or the salts thereof having the formula (3a), wherein R of formula (3a) is H or C1-C8alkyl, preferably H or C1-C6alkyl, more preferably H or C2-C4alkyl, even more preferably H, ethyl, or butyl, and in particular H.

In another preferred embodiment of the present invention, at least 50%, preferably at least 60%, more preferably at least 80%, even more preferably at least 90%, and in particular at least 95%, of the N-carbamoyl amino acid having the formula (2) is converted to L-glufosinate, its alkyl ester or the salts thereof having the formula (3b)

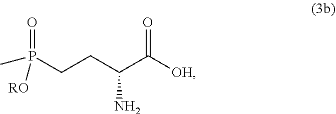

wherein R is H or C1-C8alkyl, preferably H or C1-C6alkyl, more preferably H or C2-C4alkyl, even more preferably H, ethyl, or butyl, and in particular H.

In another preferred embodiment of the present invention, 50 to 99.9%, preferably 60 to 99.8%, more preferably 80 to 99.5, even more preferably 90 to 99.2%, and in particular 95 to 99%, of the N-carbamoyl amino acid having the formula (2) is converted to L-glufosinate, its alkyl ester or the salts thereof having the formula (3b), wherein R of formula (3b) is H or C1-C8alkyl, preferably H or C1-C6alkyl, more preferably H or C2-C4alkyl, even more preferably H, ethyl, or butyl, and in particular H.

Enantiomeric excess of L-glufosinate is preferred.

In a preferred embodiment of the present invention, the cleaving is performed by an N-Carbamoyl amino acid hydrolase enzyme, preferably an L-N-Carbamoyl amino acid hydrolase enzyme. Alternatively, the cleaving can be performed by a D-N-Carbamoyl amino acid hydrolase enzyme. Suitable N-Carbamoyl amino acid hydrolase enzymes are selected from the group consisting of EC 3.5.1 Hydrolases acting on linear amides, EC 3.5.1.87 N-carbamoyl-L-amino-acid hydrolase, 3.5.1.77 N-carbamoyl-D-amino-acid hydrolase, and mixtures thereof.

Suitable N-Carbamoyl amino acid hydrolase enzymes that can be used in the method include those from Cloacibacillus sp. An23, Burkholderia lata, and the like. Suitable N-Carbamoyl amino acid hydrolase enzymes that can be used in the method include those selected from the group consisting of A0A7Y0T4N7_9RHIZ and variants thereof, Q88FQ3_PSEPK and variants thereof, Q88Q81_PSEPK and variants thereof, A0A126S6J4_PSEPU and variants thereof, Q8VUL6_9PSED and variants thereof, H9B8T5_9PSED and variants thereof, Q9FB05_9PSED and variants thereof, C0ZCM8_BREBN and variants thereof, C0Z7R5_BREB and variants thereof, A0A0K9YX84_9BACL and variants thereof, E3HUL6_ACHXA and variants thereof, A0A1V9BSS3_9BACI and variants thereof, A0A1V9BSS3_9BACI and variants thereof, Q9F464 and variants thereof, A0A4D7Q548_GEOKU and variants thereof, Q9F464 and variants thereof, A0A2S9D976_9MICC and variants thereof, A0A1I6VZZ4_9RHIZ and variants thereof, A0A1 L6RE91_9LACT and variants thereof, A0A3E0C996_9BURK and variants thereof, A0A3M7BGJ4_HORWE and variants thereof, A0A2D7YQN7_9GAMM and variants thereof, A0A535Y1H2_9CHLR and variants thereof, A0A223E415_9BACI and variants thereof, M2VSE9_GALSU and variants thereof, A0A3TOK6C0_9GAMM and variants thereof, A0A416FGE1_9CLOT and variants thereof, and variants thereof, D1P143_9GAMM and variants thereof, A0A6P2|SL4_BURL3 (SEQ ID NO:3) and variants thereof, A0A3S6Z2M9_9FIRM and variants thereof, A0A0C1 US49_9BACT and variants thereof, A0A1Y4GC62_9BACT (SEQ ID NO:2) and variants thereof, A0A3D3VMN7_9BACT and variants thereof, A0A2K8L549_9PROT and variants thereof, A0A1G0MC89_9BACT and variants thereof, A0A1M6WYS1_SELRU and variants thereof, A0A2K2BYI3_POPTR and variants thereof, A0A510DYR5_9CREN and variants thereof, A0A5Y3XFN7_SALER and variants thereof, A0A3811B54_CLODI and variants thereof, A0A2V3IQW6_9FLOR and variants thereof, *WP_035255878.1 and variants thereof, *WP_012685058.1 and variants thereof, *WP_056530217.1 and variants thereof, *WP_075270569.1 and variants thereof, and mixtures thereof, wherein variants are defined as polypeptide sequences with at least 80%, preferably 90%, and most preferably 95%, sequence identity to the respective polypeptide sequence.

In a preferred embodiment of the present invention, the cleaving is performed by an N-Carbamoyl amino acid hydrolase enzyme selected from the group consisting of Uniprot ID: A0A1Y4GC62_9BACT (SEQ ID NO:2) and variants thereof, Uniprot ID: A0A6P21SL4_BURL3 (SEQ ID NO:3) and variant thereof, Uniprot ID: A0A535Y1H2_9CHLR and variants thereof, Uniport ID: A0A3E0C996_9BURK and variants thereof, and mixtures thereof, wherein variants are defined as polypeptide sequences with at least 80%, preferably 90%, and most preferably 95%, sequence identity to the respective polypeptide sequence.

It is to be understood that the above-outlined N-Carbamoyl amino acid hydrolase enzymes are indicated in the nomenclature of the database identifier according to the Uniprot database (www.UniProt.org).

In a preferred embodiment of the present invention, R in formulae (2) and (3) is H or C1-C6alkyl, preferably H or C2-C4alkyl, more preferably H, ethyl, or butyl, and in particular H.

In a preferred embodiment of the present invention, the cleaving step is performed at a pH of 6 to 11, preferably of 6.5 to 10, more preferably of 7 to 9.5, and in particular of 7.5 to 9.

In a preferred embodiment of the present invention, the cleaving step is performed at a temperature of 20 to 50° C., preferably of 25 to 45° C., more preferably of 30 to 42° C., and in particular of 32 to 40° C.

Further, the reaction pressure is preferably ambient pressure. Preferably, the reaction pressure in the cleaving step is in the range of 0.995 to 1.030 mbar, more preferably of 1.005 to 1.020 mbar, and in particular of about 1.013 mbar.

In a preferred embodiment of the present invention, the cleaving step is performed during stirring, preferably at 50 to 1000 rpm, more preferably at 100 to 800 rpm, even more preferably at 150 to 600 rpm, still more preferably at 180 to 400 rpm, and in particular at 200 to 300 rpm.

In a preferred embodiment of the present invention, R in formulae (2) and (3) is C1-C8alkyl, preferably C1-C6alkyl, more preferably C2-C4alkyl, even more preferably ethyl or butyl, and in particular ethyl, and the method further comprises the step of
c) deprotecting under acidic conditions, preferably using hydrochloric acid or sulfuric acid.

In a preferred embodiment of the present invention, the method further comprises the addition of an N-Carbamoyl amino acid racemase enzyme. Any suitable N-Carbamoyl amino acid racemase enzyme may be possible.

The N-carbamoyl amino acid having the formula (2) can be obtained via any suitable method of manufacturing.

In a preferred embodiment, the N-carbamoyl amino acid having the formula (2) is provided by chemical synthesis. A suitable chemical approach may be starting from glufosinate (or an alkyl derivative thereof) and the addition of a cyanate such as potassium cyanate. Such a reaction preferably involves elevated temperature in the range of 35 to 80° C., preferably of 40 to 60° C., and/or reduced pressure, preferably in the range of 50 to 400 mbar, preferably 100 to 300 mbar.

In a preferred embodiment, the N-carbamoyl amino acid having the formula (2) is provided by a preceding hydrolysing step comprising hydrolysing a hydantoin having the formula (1)

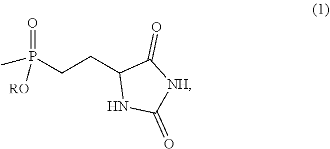

wherein R is H or C1-C8alkyl, to form the N-carbamoyl amino acid having the formula (2). The hydrolysing of the hydantoin having the formula (1) may be performed chemically or enzymatically. In a preferred embodiment, the hydantoin having the formula (1) is performed under enzymatic conditions.

In a preferred embodiment, the N-carbamoyl amino acid having the formula (2) is provided by a preceding hydrolysing step, wherein hydrolysing the hydantoin having the formula (1) is performed by a Hydantoinase enzyme.

The hydantoin having the formula (1) can be obtained via any suitable method of manufacturing. DE3142036 exemplarily discloses several synthesis.

The hydantoin having the formula (1) may exemplarily be chemically synthesized starting from an alkyl 3-cyano-3-hydroxypropyl(methyl)phosphinate such as butyl 3-cyano-3-hydroxypropyl(methyl)phosphinate, which may be treated with concentrated sulfuric acid in methanol followed by heating the mixture to a temperature above about 25° C. such as about 40° C. The obtained reaction mixture may be cooled to about 25° C. and then treated with sodium methoxide in methanol and sodium sulfate. The crude alkyl 3-cyano-3-hydroxypropyl(methyl)phosphinate may exemplarily be added to a solution of diammonium carbonate in water and the reaction mixture may be heated to a temperature of about 70° C. After standard work up, the desired alkyl hydantoin (e.g. the butyl hydantoin) can be obtained. In this connection, the alkyl may exemplarily be methyl, ethyl, propyl, butyl, pentyl, hexyl, heptyl, or octyl, preferably ethyl or butyl.

The hydantoin having the formula (1) may further exemplarily be chemically synthesized starting from a solution of glufosinate ammonium in water and potassium cyanate. After heating the reaction mixture at about 50° C. the reaction mixture can be cooled to about 25° C. followed by the addition of concentrated hydrogen chloride in water. After standard work up, the desired hydantoin can be obtained. It is further be possible to alkylate the obtained hydantoin using exemplarily triethyl orthoacetate providing the respective alkyl hydantoin (e.g. the ethyl hydantoin).

In a preferred embodiment of the present invention, the hydrolysing step is performed at a pH of 6 to 11, preferably of 6.5 to 10, more preferably of 7 to 9.5 and in particular of 7.5 to 9. The pH is preferably adjusted using alkali hydroxide, more preferably sodium hydroxide or potassium hydroxide, and in particular potassium hydroxide.

In a preferred embodiment of the present invention, the hydrolysing step is performed at a temperature of 20 to 50° C., preferably of 25 to 45° C., more preferably of 30 to 42° C., and in particular of 32 to 40° C.

In a preferred embodiment of the present invention, the hydrolysing step is performed under aqueous conditions, preferably in degassed aqueous phosphate buffer, more preferably degassed aqueous potassium phosphate buffer.

In a preferred embodiment of the present invention, the hydrolysing step is performed during stirring, preferably at 50 to 1000 rpm, more preferably at 100 to 800 rpm, even more preferably at 150 to 600 rpm, still more preferably at 180 to 400 rpm, and in particular at 200 to 300 rpm.

In another preferred embodiment, the N-carbamoyl amino acid having the formula (2) is provided by a preceding hydrolysing step, wherein hydrolysing the hydantoin having the formula (1) is performed chemically.

It is to be understood that the term “chemically” refers to reaction, which is performed under chemical conditions, i.e. not under enzymatic conditions. Hence, a chemical hydrolysing step is performed under chemical conditions, i.e. not under enzymatic condition. In general, any suitable chemical approach is possible.

A suitable chemical hydrolysing step may be performed under alkaline conditions, preferably using sodium hydroxide, potassium hydroxide or the like.

Any suitable Hydantoinase enzyme may be used.

Such Hydantoinase enzymes that can be used in the method include those from Defluviimonas alba, Rhodococcus erythropolis, Streptomyces coelicolor, Brevibacillus agri, Paenarthrobacter aurescens, Arthrobacter crystallopoietes, Bacillus sp. TS-23, Bacillus fordii, Jannaschia sp., Pseudomonas putida, Geobacillus stearothermophilus, Thermus sp., Dictyostelium discoideum, Rhizobium meliloti, Pseudomonas aeruginosa, Rhiobium radiobacter, Pseudomonas fluorescens, Glycine max, Robinia pseudoacacia, Bacillus licheniformis, Aedes aegypti, Agrobacterium fabrum, Arthrobacter sp., and the like, preferably Defluviimonas alba.

Suitable Hydantoinase enzymes are EC 3.5.2 Hydrolase acting on cyclic amides.

Further, suitable Hydantoinase enzymes may be selected from the group consisting of Q8RSQ2 and variants thereof, 069809 and variants thereof, Q846U5_9BACL and variants thereof, P81006 and variants thereof, Q84FR6_9MICC and variants thereof, Q56S49_9BACI and variants thereof, A1E351_9BAC and variants thereof, Q28SA7 and variants thereof, Q59699 and variants thereof, Q45515 and variants thereof, A0A399DRQ3_9DEIN and variants thereof, Q55DL0 and variants thereof, F7X5M8_SINMM and variants thereof, Q91676 and variants thereof, Q44184 and variants thereof, B5L363 and variants thereof, I1MEH3 and variants thereof, Q6S4R9 and variants thereof, Q65LN0 and variants thereof, Q171F8 and variants thereof, Q8U8Z6 and variants thereof, P42084 and variants thereof, Q88NW7 and variants thereof, P25995 and variants thereof, Q3Z354 and variants thereof, B1XEG2 and variants thereof, Q9F465_PAEAU and variants thereof, Q01262.1 and variants thereof, A0A250DXG4_GEOSE and variants thereof, A1SPN2 and variants thereof, Q9WYHO and variants thereof, P58329 and variants thereof, A1SGT4 and variants thereof, E3JD18 and variants thereof, HUTI_BDEBA and variants thereof, A0A161KD37_9CHLR and variants thereof, 10GL27_CALEA and variants thereof, A0A068WGW0_ECHGR and variants thereof, A0A1J4XH R4_9BACT and variants thereof, A0A1C4QIY5_9ACTN and variants thereof, A0A0K2UMP4_LEPSM and variants thereof, A0A0F5Q0A2_9RHIZ and variants thereof, A0A024KHS5_9RHIZ and variants thereof, A0A060UM69_9PROT and variants thereof, A3DKS9_STAMF and variants thereof, W2EWT0_9ACTN and variants thereof, A0A0B1T914_OESDE and variants thereof, A0A0A7LM60_9BACT and variants thereof, A0A087M7T5_9RHIZ and variants thereof, C0C180_9FIRM and variants thereof, A0A159Z531_9RHOB (see also SEQ ID NO:1) and variants thereof, R5JTP2_9CLOT and variants thereof, A0A010RM85_9PEZI and variants thereof, E1 R8C9_SEDSS and variants thereof, A0A010YEH8_9BACT and variants thereof, A0A031 LV69_9CREN and variants thereof, A0A F9QT17_9BACT and variants thereof, ALLB_BACVZ and variants thereof, HUTI_FLAPJ and variants thereof, A0A073J5J1_9BACT and variants thereof, A0A034W2Q8_BACDO and variants thereof, A0A0D8IVV8_9FIRM and variants thereof, A0A0B5QKE4_CLOBE and variants thereof, A0A098B7X6_DESHA and variants thereof, A0A0B5H4M8_9EURY and variants thereof, A0A0C1YDP1_9ACTN and variants thereof, Q981H2_RHILO and variants thereof, T1 EEH7_HELRO and variants thereof, A0A060DTG8_AZOBR and variants thereof, A0A011 MGZ5_MANHA and variants thereof, A0A060LYB6_9BACI and variants thereof, S0F3L7_CHOCR and variants thereof, A0A133VNR0_9EURY and variants thereof, A0A133U7U9_9EURY and variants thereof, A0A0U2XD52_ECOLX and variants thereof, M1YZY6_NITG3 and variants thereof, T0N9X6_9EURY and variants thereof, T0LMU2_9EURY and variants thereof, A0A0N1GBZ8_9ACTN and variants thereof, HUTI_ANASK and variants thereof, A0A031JUP0_9SPHN and variants thereof, A0A061N9L2_9BACL and variants thereof, A0A017T4D2_9DELT and variants thereof, A0A174ADZ3_9FIRM and variants thereof, A0A021X7D5_9RHIZ and variants thereof, A0A021XAC5_9RHIZ and variants thereof, A0A0C2UIW0_9BACL and variants thereof, A0A1F8NGY1_9CHLR and variants thereof, D3F1S3_CONWI and variants thereof, A0A021XG06_9RHIZ and variants thereof, U7V9Q6_9FUSO and variants thereof, D6XY37_BACIE and variants thereof, A0A0J1 FAI4_9FIRM and variants thereof, B5Y9A6_COPPD and variants thereof, PHYDA_ECOK1 and variants thereof, A0A0A9X9B7_LYGHE and variants thereof, A0A0S8H576_9BACT and variants thereof, A0A151AB14_9EURY and variants thereof, A0A064AFD7_9FUSO and variants thereof, A0A0C2FCG7_9ACTN and variants thereof, A0A0S8C148_9CHLR and variants thereof, A0A1F9CZ74_9DELT and variants thereof, A0A0A3YKD1_9ENTR and variants thereof, A0A084R4T2_STACH and variants thereof, A0A070A1Z0_9PROT and variants thereof, A0A1J4J4Y8_9EUKA and variants thereof, R1BR72_EMIHU and variants thereof, R1DD72_EMIHU and variants thereof, A0A1 L0FIA0_9ASCO and variants thereof, F7DRE9_ORNAN and variants thereof, A0LK75_SYNFM and variants thereof, A0A0Q1A918_9BACT and variants thereof, H2YZ10_CIOSA and variants thereof, 14YD99_WALMC and variants thereof, A0A077YYH5_TRITR and variants thereof, A0A077Y189_9SPHI and variants thereof, A0A089K5P4_9BACL and variants thereof, A0A0Q7W2T1_9RHIZ and variants thereof, A0A174NIK6_9FIRM and variants thereof, A0A0D5NFS5_9BACL and variants thereof, A0A0D5NNJ7_9BACL and variants thereof, A0A1H2AV66_9BACL and variants thereof, A0A0Q4RXY0_9BACL and variants thereof, A0A0Q7SB75_9BACL and variants thereof, A0A015NM92_9BACL and variants thereof, A0A100VRN2_PAEAM and variants thereof, W4BDJ0_9BACL and variants thereof, A0A147K2G0_9EURY and variants thereof, A0A0W8FVM4_9ZZZZ and variants thereof, A0A147JXR0_9EURY and variants thereof, E8R8J7_DESM0 and variants thereof, D5U113_THEAM and variants thereof, A0A1F8T9J2_9CHLR and variants thereof, G3C952_9ARCH and variants thereof, Q6YNI0_9MICC and variants thereof, A0A1G0YIQ9_9BACT and variants thereof, A0A1J5EHQ6_9DELT and variants thereof, A0A1J5E082_9DELT and variants thereof, A0A1C4PKD1_9ACTN and variants thereof, H8GX25_DEIGI and variants thereof, A0A1H5ZFN3_9BACT and variants thereof, A0A0M9Z5S1_9ACTN and variants thereof, A0A1B2HNC5_9PSEU and variants thereof, A0A1B2GNI8_STRNR and variants thereof, A0A1F8LBZ3_9CHLR and variants thereof, A0A1F8NMM2_9CHLR and variants thereof, A0A1F8SDV1_9CHLR and variants thereof, A0A1H1PLX0_9BACT and variants thereof, I0I5DC_PHYMF and variants thereof, A0A0Q5I8X4_9DEIO and variants thereof, A0A0F4JEH6_9ACTN and variants thereof, BAD75708.1, *WP_014453859.1 and variants thereof, *WP_046170519.1 and variants thereof, *CDP53201.1 and variants thereof, *WP_035078314.1 and variants thereof, *WP_042803791.1 and variants thereof, *EQB70510.1 and variants thereof, *EQB65904.1 and variants thereof, *WP_023512514.1 and variants thereof, *WP_023514195.1 and variants thereof, *WP_023516147.1 and variants thereof, *KGT87257.1 and variants thereof, *WP_045756097.1 and variants thereof, *WP_056239694.1 and variants thereof, *KU041395.1 and variants thereof, *KOV34818.1 and variants thereof, *ANZ15483.1 and variants thereof, *KJY32595.1 and variants thereof and mixtures thereof, wherein variants are defined as polypeptide sequences with at least 80%, preferably 90%, and most preferably 95%, sequence identity to the respective polypeptide sequence.

Further preferably, the Hydantoinase enzyme is selected from the group consisting of 069809 and variants thereof, Q846U5_9BACL and variants thereof, P81006 and variants thereof, Q84FR6_9MICC and variants thereof, Q56S49_9BACI and variants thereof, A1E351_9BACI and variants thereof, Q28SA7 and variants thereof, Q45515 and variants thereof, A0A399DRQ3_9DEIN and variants thereof, Q55DL0 and variants thereof, F7X5M8_SINMM and variants thereof, Q91676 and variants thereof, Q44184 and variants thereof, B5L363 and variants thereof, P42084 and variants thereof, P25995 and variants thereof, Q3Z354 and variants thereof, B1XEG2 and variants thereof, Q9F465_PAEAU and variants thereof, A0A161 KD37_9CHLR and variants thereof, A0A1J4XHR4_9BACT and variants thereof, A0A1C4QIY5_9ACTN and variants thereof, A0A0K2UMP4_LEPSM and variants thereof, A0A159Z531_9RHOB and variants thereof, E1 R8C9_SEDSS and variants thereof, A0A1F9QT17_9BACT and variants thereof, A0A0D8IVV8_9FIRM and variants thereof, A0A0B5QKE4_CLOBE and variants thereof, A0A0N1GBZ8_9ACTN and variants thereof, A0A174ADZ3_9FIRM and variants thereof, U7V9Q6_9FUSO and variants thereof, A0A0J1 FAI4_9FIRM and variants thereof, PHYDA_ECOK1 and variants thereof, A0A0S8H576_9BACT and variants thereof, A0A1J4J4Y8_9EUKA and variants thereof, A0A0D5NFS5_9BACL and variants thereof, A0A0D5NNJ7_9BACL and variants thereof, A0A1H2AV66_9BACL and variants thereof, A0A0Q4RXY0_9BACL and variants thereof, A0A0Q7SB75_9BACL and variants thereof, A0A100VRN2_PAEAM and variants thereof, W4BDJ0_9BACL and variants thereof, A0A1J5E082_9DELT and variants thereof, A0A1H5ZFN3_9BACT and variants thereof, A0A1F8NMM2_9CHLR and variants thereof, A0A1F8SDV1_9CHLR and variants thereof, A0A1H1PLX0_9BACT and variants thereof, A0A0Q5I8X4_9DEIO and variants thereof, *WP_046170519.1 and variants thereof, *WP_023514195.1 and variants thereof, *WP_023516147.1 and variants thereof, and *ANZ15483.1, and mixtures thereof, wherein variants are defined as polypeptide sequences with at least 80%, preferably 90%, and most preferably 95%, sequence identity to the respective polypeptide sequence.

Further, suitable Hydantoinase enzymes may be selected from the group consisting of, Q846U5_9BACL and variants thereof, P81006 and variants thereof, Q84FR6_9MICC and variants thereof, Q56S49_9BACI and variants thereof, Q45515 and variants thereof, A0A399DRQ3_9DEIN and variants thereof, Q55DL0 and variants thereof, F7X5M8_SINMM and variants thereof, Q91676 and variants thereof, Q44184 and variants thereof, B1XEG2 and variants thereof, A0A161KD37_9CHLR and variants thereof, A0A159Z531_9RHOB and variants thereof, E1 R8C9_SEDSS and variants thereof, A0A1F9QT179BACT and variants thereof, A0A0B5QKE4_CLOBE and variants thereof, A0A0N1GBZ8_9ACTN and variants thereof, BAD75708.1 and variants thereof, A0A064AFD7_9FUSO and variants thereof, and mixtures thereof, wherein variants are defined as polypeptide sequences with at least 80%, preferably 90%, and most preferably 95%, sequence identity to the respective polypeptide sequence.

In a preferred embodiment, the Hydantoinase enzyme is selected from the group consisting to Q846U5_9BACL and variants thereof, P81006 and variants thereof, Q84FR6_9MICC and variants thereof, A0A399DRQ3_9DEIN and variants thereof, B1XEG2 and variants thereof, A0A161KD37_9CHLR and variants thereof, A0A159Z531_9RHOB and variants thereof, E1 R8C9_SEDSS and variants thereof, A0A1F9QT17_9BACT and variants thereof, A0A0B5QKE4_CLOBE and variants thereof, A0A0N1GBZ8_9ACTN and variants thereof, BAD75708.1 and variants thereof, A0A064AFD7_9FUSO, and mixtures thereof, wherein variants are defined as polypeptide sequences with at least 80%, preferably 90%, and most preferably 95%, sequence identity to the respective polypeptide sequence.

Most preferably, the Hydantoinase enzyme is selected from the group consisting of Q45515, Q44184 and variants thereof, A0A1C4QIY5_9ACTN and variants thereof, A0A0K2UMP4_LEPSM and variants thereof, *WP_046170519.1 and variants thereof, and E1R8C9_SEDSS and variants thereof, A0A159Z531_9RHOB and variants thereof, and mixtures thereof, wherein variants are defined as polypeptide sequences with at least 80%, preferably 90%, and most preferably 95%, sequence identity to the respective polypeptide sequence.

It is to be understood that the above-outlined Hydantoinase enzymes are indicated in the nomenclature of the database identifier according to the Uniprot database (www.UniProt.org) or the NCBI protein database (www.ncbi.nlm.nih.gov/protein), where sequences from NCBI are indicated by an “*” at the beginning of the respective database identifier

In a preferred embodiment, the Hydantoinase enzyme has the SEQ ID NO:1.

In a preferred embodiment of the present invention, the Hydantoinase enzyme is an L-Hydantoinase enzyme.

In a preferred embodiment of the present invention, the Hydantoinase enzyme is a D-Hydantoinase enzyme.

In a preferred embodiment of the present invention, R in formulae (1) and (2) is H or C1-C6alkyl, preferably H or C2-C4alkyl, more preferably ethyl or butyl, and in particular ethyl.

In a preferred embodiment of the present invention, the method further comprises the addition of an Hydantoin Racemase enzyme. Any suitable Hydantoin Racemase enzyme may be possible. Suitable Hydantoin Racemase enzymes are selected from the group consisting of EC 5.1 Racemase, EC 5.1.1 Racemases acting on amino acids and derivatives, EC 5.1.99.5 Hydantoin racemase, and mixtures thereof. Suitable Hydantoin Racemase enzymes that can be used in the method include those selected from group consisting of Q9RYA6_DEIRA and variants thereof, Q9F466 and variants thereof, Q9F466 and variants thereof, A0A7L5BQP9_9RHIZ and variants thereof, Q00924 and variants thereof, F7X6X4_SINMM and variants thereof, A0A6V7ACK5_RHIRD and variants thereof, A0A7Y0XLH3_9RHIZ and variants thereof, A0A5B8XR30_9DELT and variants thereof, A0A533QH78_9PROT and variants thereof, A0A3M9Z0A0_9CYAN and variants thereof, A0A3A0A4T5_9CHLR and variants thereof, A0A1F6C9P8_HANXR and variants thereof, A0A4S0NM85_9RHIZ and variants thereof, A0A1V51086_9SPIR and variants thereof, A0A6P0NEY4_9CYAN and variants thereof, A0A2K0YBY8_9SPHN and variants thereof, A0A1H5NHN7_9RHIZ and variants thereof, A0A317KUZ3_9ACTN and variants thereof, A0A430VJ34_THESC and variants thereof, A0A1J5KHA5_9PROT and variants thereof, A0A535LIJ4_9CHLR and variants thereof, A0A2T6KHH4_9RHOB and variants thereof, A0A3G8JSD5_9ACTN and variants thereof, A0A3A9JRT3_9THEO and variants thereof, A0A2N7WBP6_9BURK and variants thereof, A0A1A2N8C4_9MYCO and variants thereof, A0A1R3TB43_9RHIZ and variants thereof, X1T733_9ZZZZ and variants thereof, A0A6P1SX79_9RHOB and variants thereof, A0A0Q5VT22_9RHIZ and variants thereof, A0A2N1RKS5_9SPIR and variants thereof, A0A529XJR5_9RHIZ and variants thereof, A0A358TXS4_9FIRM and variants thereof, A0A1Q9UJX6_9ACTN and variants thereof, A0A434WJY9_9RHIZ and variants thereof, A0A4R7C3Y1_9RHIZ and variants thereof, A0A2T4IRF7_9RHIZ and variants thereof, A0A2E8B427_9PLAN and variants thereof, A0A538D678_9ACTN and variants thereof, A0A1W6Z0D5_9BORD and variants thereof, A0A3P1 UKI1_9RHIZ and variants thereof, U2S1Q0_9FIRM and variants thereof, A0A3D5IHC5_AGRSP and variants thereof, A0A3D5JEU3_9DELT, and variants thereof, wherein variants are defined as polypeptide sequences with at least 80%, preferably 90%, and most preferably 95%, sequence identity to the respective polypeptide sequence, and mixtures thereof. It is to be understood that the above outlined Hydantoin Racemase enzymes are indicated in the nomenclature of the database identifier according to the Uniprot database (www.UniProt.org). Most preferably, the Racemase enzyme is selected from the group consisting of A0A6V7ACK5_RHIRD and variants thereof, A0A2T6KHH4_9RHOB and variants thereof, wherein variants are defined as polypeptide sequences with at least 80%, preferably 90%, and most preferably 95%, sequence identity to the respective polypeptide sequence.

In a specific embodiment of the present invention, the method comprises the addition of an Hydantoin Racemase enzyme and an N-Carbamoyl amino acid racemase enzyme.

In a preferred embodiment of the present invention, the hydrolysing step and the cleaving step are performed in a single container. In this connection, it is preferred that the hydrolysing step is performed under enzymatic conditions. Preferably, all reagents are substantially added at the start of the reaction. Alternatively, the reagents for the hydrolysing step and the reagents for the cleaving step are added to the single container at different times.

In a preferred embodiment of the present invention, the method further comprises the step of separating off a hydantoin having the formula (1b)

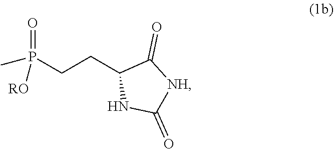

wherein R is H or C1-C8alkyl, which is obtained in hydrolysing step. Separating off the hydantoin having formula (1b) is preferably achieved using reversed phase chromatography.

Alternatively, the separation may be achieved using ion exchange, extraction, salt formation, crystallization and filtration.

The hydantoin having the formula (1b) may be chemically racemized and reused in hydrolysing step. In order to racemize hydantoins having the formula (1b) they may be treated with a suitable base, preferably at a pH of 8 or more, more preferably of 8 to 14, even more preferably of 8.5 to 12, and in particular of 8.5 to 10. Preferably the racemization is performed under aqueous conditions.

Alternatively, the hydantoin having the formula (1 b) may be treated with a Hydantoin Racemase enzyme.

In a preferred embodiment of the present invention, the method comprises the addition of a Hydantoinase enzyme, a Hydantoin Racemase enzyme, and an N-Carbamoyl amino acid hydrolase enzyme, wherein all reaction steps are performed in a single container (also known as “One-Pot” conditions), preferably wherein all reagents are substantially added at the start of the reaction or wherein the reagents are added to the single container at different times.

In another preferred embodiment of the present invention, the method comprises the addition of a Hydantoinase enzyme, a Hydantoin Racemase enzyme, an N-carbamoyl amino acid racemase enzyme, and an N-Carbamoyl amino acid hydrolase enzyme, wherein all reaction steps are performed in a single container (also known as “One-Pot” conditions), preferably wherein all reagents are substantially added at the start of the reaction or wherein the reagents are added to the single container at different times.

In another preferred embodiment of the present invention, the method comprises the addition of an N-carbamoyl amino acid racemase enzyme and an N-Carbamoyl amino acid hydrolase enzyme, wherein all reaction steps are performed in a single container (also known as “One-Pot” conditions), preferably wherein all reagents are substantially added at the start of the reaction or wherein the reagents are added to the single container at different times.

In yet another preferred embodiment of the present invention, the method comprises the addition of a Hydantoinase enzyme and an N-Carbamoyl amino acid hydrolase enzyme, wherein all reaction steps are performed in a single container (also known as “One-Pot” conditions), preferably wherein all reagents are substantially added at the start of the reaction or wherein the reagents are added to the single container at different times. In this connection it is preferred if the pH of the reaction mixture is of 7 to 9.

The applied enzymes may be applied via any suitable known in the art way.

In a preferred embodiment of the present invention, the applied enzymes are applied as cleared cell lysate, whole cells, or immobilized enzymes.

Alternatively, some or all of the components other than L-glufosinate can be removed from the biotransformation mixture, the mixture optionally concentrated, and then the mixture can be used directly (and/or with the addition of various adjuvants) for the prevention or control of weeds. The biotransformation mixture, in some instances, can be used directly (and/or with the addition of various adjuvants) for the prevention or control of weeds.

Additional steps to further purify the L-glufosinate can be added. Such further purification and isolation methods include ion exchange, extraction, salt formation, crystallization, and filtration; each may be used multiple times or in suitable combination. Enzymes can be removed by simple filtration if supported, or if free in solution by the use of ultrafiltration, the use of absorbants like celite, cellulose or carbon, or denaturation via various techniques known to those skilled in the art.

Ion exchange processes effect separation by selective adsorption of solutes onto resins chosen for this purpose. Because products and impurities must be dissolved in a single solution prior to adsorption, concentration of the purified product stream by evaporation or distillation prior to isolation is usually required. Examples of the use of ion exchange for purification are described by Schultz et al., and in EP0249188(A2).

Purification may be achieved by the formation of an insoluble salt of L-glufosinate by the addition of a suitable acid, including hydrochloric acid, sulfuric acid, phosphoric acid, nitric acid, acetic acid and the like. Similarly, the purification may be achieved by the addition of a suitable base to form an insoluble salt. Useful bases include hydroxides, carbonates, sulfates and phosphates of alkali metals or hydroxides, carbonates, sulfates and phosphates of alkali earth metals. Other bases which contain nitrogen may be used, including ammonia, hydroxylamine, isopropylamine, triethylamine, tributylamine, pyridine, 2-picoline, 3-picoline, 4-picoline, 2,4-lutidine, 2,6-lutidine, morpholine, N-methymorpholine, 1,8-diazabicyclo[5.4.0]undec-7-ene, and dimethylethanolamine. It may be advantageous to concentrate the mixture or to add a solvent (or both) to maximize yield and optimize purity of the desired salt. Solvents suitable for this purpose include those in which the solubility of the desired salt is very low (such solvents are often called “anti-solvents”). Salts of L-glufosinate can be transformed into forms of glufosinate suitable for formulation by standard methods known to those skilled in the art. Alternatively, the L-glufosinate can be isolated as a zwitterion.

U.S. Pat. No. 9,255,115 B2 describes how the hydrochloric acid salt of L-glufosinate can be converted to the zwitterionic form with a base such as sodium hydroxide or sodium methoxide and then crystallized from aqueous alcohol solvent to afford L-glufosinate in relatively high purity. This method has the advantage of producing crystalline L-glufosinate that is not hygroscopic and therefore maintains a higher purity compared to amorphous L-glufosinate when exposed to humidity over time.

Other salts of L-glufosinate are known in the art. U.S. Pat. Nos. 5,767,309 and 5,869,668 teach the use of chiral alkaloid bases to form diastereomeric salts with racemic glufosinate. Purification is achieved because the salt of L-glufosinate precipitates from solution in much larger quantity than the corresponding salt of D-glufosinate. Therefore this method could be used with the present invention to obtain L-glufosinate with high enantiomeric excess, if desired.

Optionally, purification may be achieved by first crystallizing one or more impurities, removing the impurities by filtration and then further purifying L-glufosinate from the resulting filtrate by forming a salt as previously described. This is advantageous if unreacted amine donor can be partially or completely isolated and used in subsequent reactions. Similarly, unreacted N-carbamoyl amino acid having the formula (2) that is partially or completely isolated may be recycled for use in subsequent reactions.

Extraction may be used to purify the product. DE 3920570 C2 describes a process in which excess glutamic acid (used as the amine donor) is precipitated by adjusting the solution pH to 3.7 to 4.2 with sulfuric acid. After filtering the glutamic acid, the filtrate pH is lowered to 1-2 whereupon other impurities are extracted into a solvent. After extraction and concentration, ammonia is added to the aqueous solution to a pH of 5-7 whereupon ammonium sulfate precipitates. The ammonium sulfate is removed by filtration and the resulting filtrate is concentrated to afford the ammonium salt of L-glufosinate.

Isolation of L-glufosinate or its salts may be desirable, for example, for the purpose of shipping solids to the location of formulation or use. Typical industrial methods of isolation may be used, for example, a filtration, centrifugation, etc. Isolated product often requires the removal of water, volatile impurities and solvents (if present) and typical industrial drying equipment may be used for this purpose. Examples of such equipment include ovens, rotating drum dryers, agitated dryers, etc. In some cases, it may be advantageous to use a spray dryer.

It is not necessary to produce a solid product after purification. This may be advantageous if the formulation of L-glufosinate is to occur at the same site used for L-glufosinate production. L-glufosinate and many of its salts are readily soluble in water, and water is a convenient liquid to use for formulating products. For example, the amine donor is isolated by filtration and the resulting filtrate is concentrated by distillation. The pH of the filtrate may be adjusted to a desirable value and the resulting solution may be used as is or blended with formulation ingredients. In another example, a slurry of L-glufosinate or one of its salts may be prepared as described above and isolated by filtration. The solid could be dissolved directly on the filter by adding water or a suitable solvent to obtain a solution of L-glufosinate.

Further, a method of manufacturing glufosinate, its alkyl ester or the salts thereof having the formula (3)

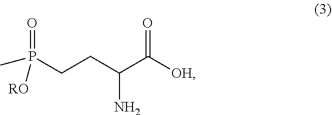

wherein R is H or C1-C8alkyl, comprising the step of chemically cleaving off the carbamoyl moiety of a N-carbamoyl amino acid having the formula (2)

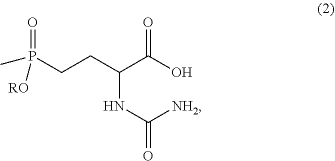

wherein R is H or C1-C8alkyl, is disclosed.

It is to be understood that the term “chemically cleaving” refers to a cleaving step that is not performed under enzymatic conditions. Any suitable chemical approach is possible.

Preferably, the chemically cleaving is performed under acidic conditions, preferably using hydrogen chloride, sulfuric acid, and mixtures thereof, more preferably hydrogen chloride, and in particular concentrated hydrogen chloride (i.e. 34 to 38% of hydrogen chloride in water).

Preferably, the chemically cleaving is performed by adding sodium nitrite.

Preferably, the chemically cleaving is performed at elevated temperature, more preferably at a temperature of 50 to 130° C., even more preferably of 70 to 120° C., and in particular of 80 to 110° C.

Preferably, the reaction pressure is ambient pressure. The reaction pressure is preferably in the range of 0.995 to 1.030 mbar, more preferably of 1.005 to 1.020 mbar, and in particular of about 1.013 mbar.

In a preferred embodiment of the present invention, the cleaving is performed at a pH of 0 to 5, preferably of 0 to 3.

In a particular embodiment, the chemically cleaving is performed by i) dissolving the N-carbamoyl amino acid having the formula (2) under acidic conditions, preferably at a temperature of −15 to 20° C., more preferably of −10 to 10° C., and in particular of −5 to 5° C., ii) addition of sodium nitrite, preferably following by stirring at a temperature of 10 to 35° C., preferably of 15 to 30° C., and in particular of 20 to 25° C. or room temperature, and optionally iii) adding an acid, preferably concentrated hydrogen chloride, and heating the reaction mixture to a temperature of 50 to 130° C., preferably of 70 to 120° C., and in particular of 80 to 110° C. Step iii) is suitable to cleave off the ester, if present.

The reaction mixture can be worked-up under standard procedure (i.e. washing and purifying).

As mentioned above, the invention further relates in a second aspect to a composition comprising a N-carbamoyl amino acid having the formula (2a)

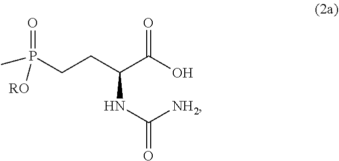

wherein R is H or C1-C8alkyl, and L-glufosinate or the salts thereof.

In a preferred embodiment of the present invention, R in formula (2a) is H or C1-C6alkyl, preferably H or C2-C4alkyl, more preferably H, ethyl or butyl, and in particular H.

Suitable salts are hydrochloric acid salt, ammonium salts, and isopropylammonium salts. It is further to be understood that the respective zwitterion of L-glufosinate is also encompassed.

In a preferred embodiment of the present invention, the amount of L-glufosinate or the salts thereof is at least 30 wt.-%, preferably at least 40 wt.-%, more preferably at least 50 wt.-%, even more preferably at least 60 wt.-%, still more preferably at least 70 wt.-%, and in particular at least 80 wt.-% or at least 90 wt.-%, based on the total amount of the N-carbamoyl amino acid having the formula (2a) and L-glufosinate or the salts thereof.

In a preferred embodiment of the present invention, the amount of L-glufosinate or the salts thereof is in the range of 20 to 99.9 wt.-%, preferably of 30 to 99.8 wt.-%, more preferably of 40 to 99.7 wt.-%, even more preferably of 50 to 99.6 wt.-%, still more preferably of 60 to 99.5 wt.-%, and in particular of 70 to 99.5 wt.-% or of 80 to 99.5 wt.-%, based on the total amount of the N-carbamoyl amino acid having the formula (2a) and L-glufosinate or the salts thereof.

In a preferred embodiment of the present invention, the amount of L-glufosinate or the salts thereof is in the range of 80 to 99.995 wt.-%, preferably of 90 to 99.99 wt.-%, more preferably of 95 to 99.95 wt.-%, even more preferably of 97 to 99.92 wt.-%, and in particular of 99 to 99.9 wt.-%, based on the total amount of the N-carbamoyl amino acid having the formula (2a) and L-glufosinate or the salts thereof.

The composition can comprise the N-carbamoyl amino acid having the formula (2a)

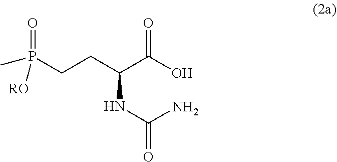

in an amount of up to 70 wt.-%, preferably up to 60 wt.-%, more preferably up to 50 wt.-%, even more preferably up to 40 wt.-%, still more preferably up to 30 wt.-%, and in particular up to 20 wt.-% or up to 10 wt.-%, based on the total amount of the N-carbamoyl amino acid having the formula (2a) and L-glufosinate or the salts thereof. The composition can also comprise the N-carbamoyl amino acid having the formula (2a) in an amount of up to 40 wt.-%, preferably up to 20 wt.-%, more preferably up to 10 wt.-%, even more preferably up to 5 wt.-%, still more preferably up to 3 wt.-%, and in particular up to 1 wt.-%, based on the total amount of the N-carbamoyl amino acid having the formula (2a) and L-glufosinate or the salts thereof.

The composition can comprise the N-carbamoyl amino acid having the formula (2a)

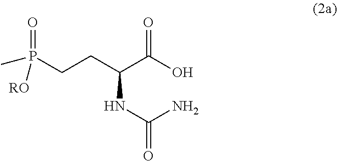

in an amount of 0.1 zo 80 wt.-%, preferably of 0.2 to 70 wt.-%, more preferably of 0.3 to 60 wt.-%, even more preferably of 0.4 to 50 wt.-%, still more preferably of 0.5 to 40 wt.-%, and in particular 0.5 to 30 wt.-% or 0.5 to 20 wt.-%, based on the total amount of the N-carbamoyl amino acid having the formula (2a) and L-glufosinate or the salts thereof. The composition can also comprise the N-carbamoyl amino acid having the formula (2a) in an amount of 0.001 to 40 wt.-%, preferably 0.005 to 20 wt.-%, more preferably 0.01 to 10 wt.-%, even more preferably 0.05 to 5 wt.-%, still more preferably 0.08 to 3 wt.-%, and in particular 0.1 to 1 wt.-%, based on the total amount of the N-carbamoyl amino acid having the formula (2a) and L-glufosinate or the salts thereof.

The composition can further comprise the N-carbamoyl amino acid having the formula (2b)

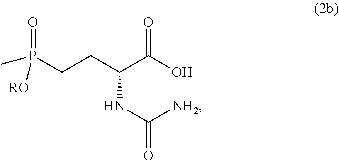

preferably in an amount of up to 40 wt.-%, preferably up to 20 wt.-%, more preferably up to 10 wt.-%, even more preferably up to 5 wt.-%, still more preferably up to 3 wt.-%, and in particular up to 1 wt.-%, based on the total amount of the N-carbamoyl amino acid having the formula (2a), the N-carbamoyl amino acid having the formula (2b), and L-glufosinate or the salts thereof.

The composition can further comprise the N-carbamoyl amino acid having the formula (2b)

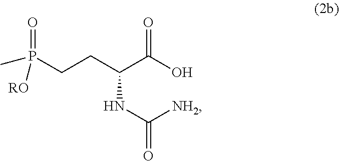

in an amount of 0.001 to 40 wt.-%, preferably 0.005 to 20 wt.-%, more preferably 0.01 to 10 wt.-%, even more preferably 0.05 to 5 wt.-%, still more preferably 0.08 to 3 wt.-%, and in particular 0.5 to 1 wt.-%, based on the total amount of the N-carbamoyl amino acid having the formula (2a), the N-carbamoyl amino acid having the formula (2b), and L-glufosinate or the salts thereof.

The composition can further comprise a hydantoin having the formula (1b)

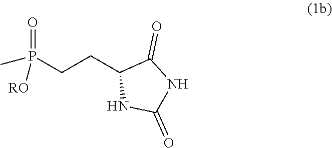

and/or
a hydantoin having the formula (1a)

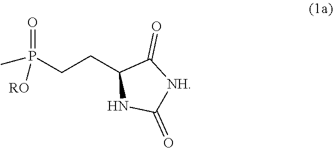

The hydantoin (having the formulae (1b) and/or (1a)) is preferably present in the composition in an amount of up to 30 wt.-%, preferably up to 20 wt.-%, more preferably up to 10 wt.-%, even more preferably up to 5 wt.-%, still more preferably up to 2 wt.-%, and in particular up to 0.5 wt.-%, based on the total amount of the hydantoin (having the formulae (1b) and/or (1a)), the N-carbamoyl amino acid having the formula (2a), and L-glufosinate or the salts thereof.

Alternatively, the hydantoin (having the formulae (1b) and/or (1a)) is present in the composition in an amount of 0.001 to 30 wt.-%, preferably 0.005 to 20 wt.-%, more preferably 0.01 to 10 wt.-%, even more preferably 0.05 to 5 wt.-%, still more preferably 0.08 to 2 wt.-%, and in particular 0.1 to 0.5 wt.-%, based on the total amount of the hydantoin (having the formulae (1b) and/or (1a)), the N-carbamoyl amino acid having the formula (2a), and L-glufosinate or the salts thereof.

In a preferred embodiment of the present invention, R in formulae (2a) and (1b) is H or C1-C6alkyl, preferably H or C2-C4alkyl, more preferably H, ethyl or butyl, and in particular H or ethyl, if present. In this connection it is to be understood that R in formulae (2b) and (1a) is also preferably H or C1-C6alkyl, preferably H or C2-C4alkyl, more preferably H, ethyl or butyl, and in particular H or ethyl, if present. In this connection it is to be understood that R in formulae (2b), (1a), and (1b) is preferably H or C1-C6alkyl, preferably H or C2-C4alkyl, more preferably H, ethyl or butyl, and in particular H or ethyl, if present.

In one preferred embodiment of the present invention, the herein described composition may be used directly as a herbicidal compositions or as an ingredient in a formulated herbicidal product.

The compositions described herein are useful for application to a field of crop plants for the prevention or control of weeds. The composition may be formulated as a liquid for spraying on a field. The glufosinate, preferably the L-glufosinate, is provided in the composition in effective amounts. As used herein, effective amount means from about 10 grams active ingredient per hectare to about 1,500 grams active ingredient per hectare, e.g., from about 50 grams to about 400 grams or from about 100 grams to about 350 grams. In some embodiments, the active ingredient is L-glufosinate. For example, the amount of L-glufosinate in the composition can be about 10 grams, about 50 grams, about 100 grams, about 150 grams, about 200 grams, about 250 grams, about 300 grams, about 350 grams, about 400 grams, about 500 grams, about 550 grams, about 600 grams, about 650 grams, about 700 grams, about 750 grams, about 800 grams, about 850 grams, about 900 grams, about 950 grams, about 1,000 grams, about 1,050 grams, about 1,100 grams, about 1,150 grams, about 1,200 grams, about 1,250 grams, about 1,300 grams, about 1,350 grams, about 1,400 grams, about 1,450 grams, or about 1,500 grams L-glufosinate per hectare.

The herbicidal compositions (including concentrates which require dilution prior to application to the plants) described herein contain L-glufosinate (i.e., the active ingredient), optionally

N-carbamoyl amino acid having the formula (2a)

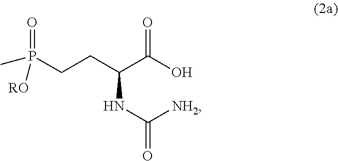

and one or more adjuvant components in liquid or solid form.

The composition may also comprise a herein further described hydantoin of formula (1).

The compositions are prepared by admixing the active ingredient with one or more adjuvants, such as diluents, extenders, carriers, surfactants, organic solvents, humectants, or conditioning agents, to provide a composition in the form of a finely-divided particulate solid, pellet, solution, dispersion, or emulsion. Thus, the active ingredient can be used with an adjuvant, such as a finely-divided solid, a liquid of organic origin, water, a wetting agent, a dispersing agent, an emulsifying agent, or any suitable combination of these. From the viewpoint of economy and convenience, water is the preferred diluent. However, not all the compounds are resistant to hydrolysis and in some cases this may dictate the use of non-aqueous solvent media, as understood by those of skill in the art.

Optionally, one or more additional components can be added to the composition to produce a formulated herbicidal composition. Such formulated compositions can include L-glufosinate, carriers (e.g., diluents and/or solvents), and other components. The formulated composition includes an effective amount of L-glufosinate.

A diluent can also be included in the formulated composition. Suitable diluents include water and other aqueous components. Optionally, the diluents are present in an amount necessary to produce compositions ready for packaging or for use.

The herbicidal compositions described herein, particularly liquids and soluble powders, can contain as further adjuvant components one or more surface-active agents in amounts sufficient to render a given composition readily dispersible in water or in oil. The incorporation of a surface-active agent into the compositions greatly enhances their efficacy. Surface-active agent, as used herein, includes wetting agents, dispersing agents, suspending agents, and emulsifying agents are included therein. Anionic, cationic, and non-ionic agents can be used with equal facility.

Suitable wetting agents include alkyl benzene and alkyl naphthalene sulfonates, sulfated fatty alcohols, amines or acid amides, long chain acid esters of sodium isothionate, esters of sodium sulfosuccinate, sulfated or sulfonated fatty acid esters petroleum solfonates, sulfonated vegetable oils, ditertiary acetylenic glycols, polyoxyethylene derivatives of alkylphenols (particularly isooctylphenol and nonylphenol), and polyoxethylene derivatives of the mono-higher fatty acid esters of hexitol anhydrides (e.g. sorbitan). Exemplary dispersants include methyl cellulose, polyvinyl alcohol, sodium lignin sulfonates, polymeric alkyl naphthalene sulfonates, sodium naphthalene sulfonate, polymethylene bisnaphthalenesulfonate, and sodium N-methyl-N- (long chain acid) laurates.

Water-dispersible powder compositions can be made containing one or more active ingredients, an inert solid extender, and one or more wetting and dispersing agents. The inert solid extenders are usually of mineral origin, such as the natural clays, diatomaceous earth, and synthetic minerals derived from silica and the like. Examples of such extenders include kaolinites, attapulgite clay, and synthetic magnesium silicate. Water-dispersible powders described herein can optionally contain from about 5 to about 95 parts by weight of active ingredient (e.g., from about 15 to 30 parts by weight of active ingredient), from about 0.25 to 25 parts by weight of wetting agent, from about 0.25 to 25 parts by weight of dispersant, and from 4.5 to about 94.5 parts by weight of inert solid extender, all parts being by weight of the total composition. Where required, from about 0.1 to 2.0 parts by weight of the solid inert extender can be replaced by a corrosion inhibitor or anti-foaming agent or both.

Aqueous suspensions can be prepared by dissolution or by mixing together and grinding an aqueous slurry of a water-insoluble active ingredient in the presence of a dispersing agent to obtain a concentrated slurry of very finely-divided particles. The resulting concentrated aqueous suspension is characterized by its extremely small particle size, so that when diluted and sprayed, coverage is very uniform.

Emulsifiable oils are usually solutions of active ingredient in water-immiscible or partially water-immiscible solvents together with a surface active agent. Suitable solvents for the active ingredient described herein include hydrocarbons and water-immiscible ethers, esters, or ketones. The emulsifiable oil compositions generally contain from about 5 to 95 parts active ingredient, about 1 to 50 parts surface active agent, and about 4 to 94 parts solvent, all parts being by weight based on the total weight of emulsifiable oil.

Compositions described herein can also contain other additaments, for example, fertilizers, phytotoxicants and plant growth regulants, pesticides, and the like used as adjuvants or in combination with any of the above-described adjuvants. The compositions described herein can also be admixed with the other materials, e.g., fertilizers, other phytotoxicants, etc., and applied in a single application.

In each of the formulation types described herein, e.g., liquid and solid formulations, the concentration of the active ingredients are the same.

It is recognized that the herbicidal compositions can be used in combination with other herbicides. The herbicidal compositions of the present invention are often applied in conjunction with one or more other herbicides to control a wider variety of undesirable vegetation. When used in conjunction with other herbicides, the presently claimed compounds can be formulated with the other herbicide or herbicides, tank mixed with the other herbicide or herbicides or applied sequentially with the other herbicide or herbicides. Some of the herbicides that can be employed in conjunction with the compounds of the present invention include: amide herbicides such as allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid and tebutam; anilide herbicides such as chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen and propanil; arylalanine herbicides such as benzoylprop, flamprop and flamprop-M; chloroacetanilide herbicides such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor and xylachlor; sulfonanilide herbicides such as benzofluor, perfluidone, pyrimisulfan and profluazol; sulfonamide herbicides such as asulam, carbasulam, fenasulam and oryzalin; antibiotic herbicides such as bilanafos; benzoic acid herbicides such as chloramben, dicamba, 2,3,6-TBA and tricamba; pyrimidinyloxybenzoic acid herbicides such as bispyribac and pyriminobac; pyrimidinylthiobenzoic acid herbicides such as pyrithiobac; phthalic acid herbicides such as chlorthal; picolinic acid herbicides such as aminopyralid, clopyralid and picloram; quinolinecarboxylic acid herbicides such as quinclorac and quinmerac; arsenical herbicides such as cacodylic acid, CMA, DSMA, hexaflurate, MAA, MAMA, MSMA, potassium arsenite and sodium arsenite; benzoylcyclohexanedione herbicides such as mesotrione, sulcotrione, tefuryltrione and tembotrione; benzofuranyl alkylsulfonate herbicides such as benfuresate and ethofumesate; carbamate herbicides such as asulam, carboxazole chlorprocarb, dichlormate, fenasulam, karbutilate and terbucarb; carbanilate herbicides such as barban, BCPC, carbasulam, carbetamide, CEPC, chlorbufam, chlorpropham, CPPC, desmedipham, phenisopham, phenmedipham, phenmedipham-ethyl, propham and swep; cyclohexene oxime herbicides such as alloxydim, butroxydim, clethodim, cloproxydim, cycloxydim, profoxydim, sethoxydim, tepraloxydim and tralkoxydim; cyclopropylisoxazole herbicides such as isoxachlortole and isoxaflutole; dicarboximide herbicides such as benzfendizone, cinidon-ethyl, flumezin, flumiclorac, flumioxazin and flumipropyn; dinitroaniline herbicides such as benfluralin, butralin, dinitramine, ethalfluralin, fluchloralin, isopropalin, methalpropalin, nitralin, oryzalin, pendimethalin, prodiamine, profluralin and trifluralin; dinitrophenol herbicides such as dinofenate, dinoprop, dinosam, dinoseb, dinoterb, DNOC, etinofen and medinoterb; diphenyl ether herbicides such as ethoxyfen; nitrophenyl ether herbicides such as acifluorfen, aclonifen, bifenox, chlomethoxyfen, chlomitrofen, etnipromid, fluorodifen, fluoroglycofen, fluoronitrofen, fomesafen, furyloxyfen, halosafen, lactofen, nitrofen, nitrofluorfen and oxyfluorfen; dithiocarbamate herbicides such as dazomet and metam; halogenated aliphatic herbicides such as alorac, chloropon, dalapon, flupropanate, hexachloroacetone, iodomethane, methyl bromide, monochloroacetic acid, SMA and TCA; imidazolinone herbicides such as imazamethabenz, imazamox, imazapic, imazapyr, imazaquin and imazethapyr; inorganic herbicides such as ammonium sulfamate, borax, calcium chlorate, copper sulfate, ferrous sulfate, potassium azide, potassium cyanate, sodium azide, sodium chlorate and sulfuric acid; nitrile herbicides such as bromobonil, bromoxynil, chloroxynil, dichlobenil, iodobonil, ioxynil and pyraclonil; organophosphorus herbicides such as amiprofos-methyl, anilofos, bensulide, bilanafos, butamifos, 2,4-DEP, DMPA, EBEP, fosamine, glyphosate and piperophos; phenoxy herbicides such as bromofenoxim, clomeprop, 2,4-DEB, 2,4-DEP, difenopenten, disul, erbon, etnipromid, fenteracol and trifopsime; phenoxyacetic herbicides such as 4-CPA, 2,4-D, 3,4-DA, MCPA, MCPA-thioethyl and 2,4,5-T; phenoxybutyric herbicides such as 4-CPB, 2,4-DB, 3,4-DB, MCPB and 2,4,5-TB; phenoxypropionic herbicides such as cloprop, 4-CPP, dichlorprop, dichlorprop-P, 3,4-DP, fenoprop, mecoprop and mecoprop-P; aryloxyphenoxypropionic herbicides such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P and trifop; phenylenediamine herbicides such as dinitramine and prodiamine; pyrazolyl herbicides such as benzofenap, pyrazolynate, pyrasulfotole, pyrazoxyfen, pyroxasulfone and topramezone; pyrazolylphenyl herbicides such as fluazolate and pyraflufen; pyridazine herbicides such as credazine, pyridafol and pyridate; pyridazinone herbicides such as brompyrazon, chloridazon, dimidazon, flufenpyr, metflurazon, norflurazon, oxapyrazon and pydanon; pyridine herbicides such as aminopyralid, cliodinate, clopyralid, dithiopyr, fluroxypyr, haloxydine, picloram, picolinafen, pyriclor, thiazopyr and triclopyr; pyrimidinediamine herbicides such as iprymidam and tioclorim; quaternary ammonium herbicides such as cyperquat, diethamquat, difenzoquat, diquat, morfamquat and paraquat; thiocarbamate herbicides such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate and vemolate; thiocarbonate herbicides such as dimexano, EXD and proxan; thiourea herbicides such as methiuron; triazine herbicides such as dipropetryn, triaziflam and trihydroxytriazine; chlorotriazine herbicides such as atrazine, chlorazine, cyanazine, cyprazine, eglinazine, ipazine, mesoprazine, procyazine, proglinazine, propazine, sebuthylazine, simazine, terbuthylazine and trietazine; methoxytriazine herbicides such as atraton, methometon, prometon, secbumeton, simeton and terbumeton; methylthiotriazine herbicides such as ametryn, aziprotryne, cyanatryn, desmetryn, dimethametryn, methoprotryne, prometryn, simetryn and terbutryn; triazinone herbicides such as ametridione, amibuzin, hexazinone, isomethiozin, metamitron and metribuzin; triazole herbicides such as amitrole, cafenstrole, epronaz and flupoxam; triazolone herbicides such as amicarbazone, bencarbazone, carfentrazone, flucarbazone, propoxycarbazone, sulfentrazone and thiencarbazone-methyl; triazolopyrimidine herbicides such as cloransulam, diclosulam, florasulam, flumetsulam, metosulam, penoxsulam and pyroxsulam; uracil herbicides such as butafenacil, bromacil, flupropacil, isocil, lenacil and terbacil; 3-phenyluracils; urea herbicides such as benzthiazuron, cumyluron, cycluron, dichloralurea, diflufenzopyr, isonoruron, isouron, methabenzthiazuron, monisouron and noruron; phenylurea herbicides such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluron, phenobenzuron, siduron, tetrafluron and thidiazuron; pyrimidinylsulfonylurea herbicides such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron and trifloxysulfuron; triazinylsulfonylurea herbicides such as chlorsulfuron, cinosulfuron, ethametsulfuron, iodosulfuron, metsulfuron, prosulfuron, thifensulfuron, triasulfuron, tribenuron, triflusulfuron and tritosulfuron; thiadiazolylurea herbicides such as buthiuron, ethidimuron, tebuthiuron, thiazafluron and thidiazuron; and unclassified herbicides such as acrolein, allyl alcohol, aminocyclopyrachlor, azafenidin, benazolin, bentazone, benzobicyclon, buthidazole, calcium cyanamide, cambendichlor, chlorfenac, chlorfenprop, chlorflurazole, chlorflurenol, cinmethylin, clomazone, CPMF, cresol, ortho-dichlorobenzene, dimepiperate, endothal, fluoromidine, fluridone, flurochloridone, flurtamone, fluthiacet, indanofan, methazole, methyl isothiocyanate, nipyraclofen, OCH, oxadiargyl, oxadiazon, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan and tritac. The herbicidal compositions of the present invention can, further, be used in conjunction with glyphosate or 2,4-D on glyphosate-tolerant or 2,4-D-tolerant crops. It is generally preferred to use the compositions of the invention in combination with herbicides that are selective for the crop being treated and which complement the spectrum of weeds controlled by these compositions at the application rate employed. It is further generally preferred to apply the compositions of the invention and other complementary herbicides at the same time, either as a combination formulation or as a tank mix.

As mentioned above, the invention further relates in a third aspect to a method for selectively controlling weeds in an area, preferably containing a crop of planted seeds or crops that are resistant to glufosinate, comprising:
applying an effective amount of a composition comprising L-glufosinate or the salts thereof at an enantiomeric proportion of at least 80% over D-glufosinate or the salts thereof and more than 0.01 wt.-% to less than 10 wt.-%, based on the total amount of the composition, of a N-carbamoyl amino acid having the formula (2)

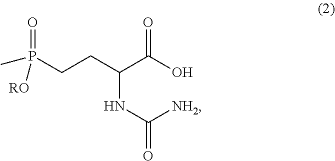

wherein R is H or C1-C8alkyl, to the area.

In a preferred embodiment of the present invention, the composition comprises L-glufosinate or the salts thereof at an enantiomeric proportion of 80 to 99.9%, preferably in an enantiomeric proportion of 85 to 99.8%, more preferably of 90 to 99.7%, and in particular of 92 to 99.5%, over D-glufosinate or the salts thereof.

In a preferred embodiment of the present invention, the composition comprises 0.02 to 8 wt.-%, preferably 0.03 to 5 wt.-%, more preferably 0.05 to 3 wt.-%, and in particular 0.1 to 2 wt.-%, based on the total amount of the composition, of a N-carbamoyl amino acid having the formula (2)

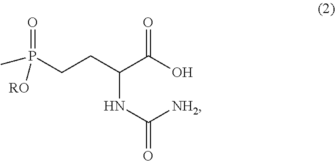

wherein R is H or C1-C8alkyl, preferably H.

It is to be understood that the composition may comprise the same adjuvants and/or other herbicides as described in more detail above.

Preferred embodiments are above-described in further detail.

The compositions described herein are useful for application to a field of crop plants for the prevention or control of weeds. The composition may be formulated as a liquid for spraying on a field. The L-glufosinate is provided in the composition in effective amounts. As used herein, effective amount means from about 10 grams active ingredient per hectare to about 1,500 grams active ingredient per hectare, e.g., from about 50 grams to about 400 grams or from about 100 grams to about 350 grams. In some embodiments, the active ingredient is L-glufosinate. For example, the amount of L-glufosinate in the composition can be about 10 grams, about 50 grams, about 100 grams, about 150 grams, about 200 grams, about 250 grams, about 300 grams, about 350 grams, about 400 grams, about 500 grams, about 550 grams, about 600 grams, about 650 grams, about 700 grams, about 750 grams, about 800 grams, about 850 grams, about 900 grams, about 950 grams, about 1,000 grams, about 1,050 grams, about 1,100 grams, about 1,150 grams, about 1,200 grams, about 1,250 grams, about 1,300 grams, about 1,350 grams, about 1,400 grams, about 1,450 grams, or about 1,500 grams L-glufosinate per hectare.

The present invention is further illustrated by the following examples.

### Examples

### Material & Methods

### Preparation of Enzymes

### a) Cloning of Enzyme Genes (Ex 1)

The amino acid sequences of the respective enzymes were identified from public databases (UniProt, https://www.uniprot.org; NCBI protein database, https://www.ncbi.nlm.nih.gov/protein. Sequences from NCBI are indicated by an “*” at the beginning of the respective database identifier). The respective DNA sequence was derived thereof using standard codon usage of Escherichia coli. The DNA sequence was synthesized (BioCat GmbH) and cloned into the plasmid pDHE19.2 (Ress-Loeschke, M. et al., DE 19848129, 1998, (BASF AG)). The resulting plasmids were used to transform competent cells (Chung, C. T. et al., Proc Natl Acad Sci USA, 1989, 86, 2172) of the E. coli strain TG10, pAgro, pHSG575 (E. coli TG10 (Kesseler, M. et al., WO2004050877A1, 2004, (BASF AG)):rhaA- -derivate of E. coli TG1 transformed with pHSG575 (Takeshita, S. et al., Gene, 1987, 61, 63) and pAgro4 (pBB541 in Tomoyasu, T. et al., Mol. Microbiol., 2001, 40, 397).

### b) Recombinant Production of Enzymes (Ex 2)

### Biocatalyst Preparation in Shake Flasks

E. coli TG10 carrying the recombinant plasmid of the enzyme was used to inoculate 2 ml LB medium (Bertani, G., J Bacteriol, 1951, 62, 293) supplemented with 100 μg/l ampicillin, 100 μg/l spectinomycin, 20 μg/l chloramphenicol and the resulting pre-culture was incubated for 5 h at 37° C. at an agitation of 250 rpm. 1 ml of the pre-culture was used to inoculate 100 ml LB medium supplemented with 100 μg/l ampicillin, 100 μg/l spectinomycin, 20 μg/l chloramphenicol, 1 mM MnCl2, 0.1 mM isopropyl-ß-D-thiogalactopyranosid, and 0.5 g/I rhamnose in a 500 ml baffled Erlenmeyer-flask. The culture was incubated at 37° C. for 18 h under shaking conditions. Subsequently, the biomass was harvested by centrifugation at 3220×g for 10 min at 8° C. The supernatant was discarded, and the cell pellet resuspended in 8 ml H EPES buffer at a concentration of 100 mM and pH 8.2 supplemented with 1 mM MnCl2. The cell suspension was used without any further preparation for synthesis in case whole cell biotransformation were carried out. In case cleared cell lysates were employed instead, 5 ml of the cell suspension were distributed into 5 reaction tubes containing lysing matrix B (0.7 ml quartz-beads at Ø 0.1 mm, MP Biomedicals), the tubes chilled on ice, and cells subsequently broken in a homogenizer (Peqlab Precellys24, VWR) for two 30 second cycles. In between cycles samples were chilled on ice. The resulting cell free lysates were cleared by centrifugation 20817×g for 10 min, at 8° C. The supernatants were isolated and fractions from the same batch combined (=cleared cell lysate).

### Fermentative Whole-Cell Biocatalyst Production

E. coli TG10 containing the plasmids pAgro4 and pHSG575 were transformed with pDHE plasmid encoding the protein of interest. Transformants were cultivated on a LB agar plate supplemented with 100 μg/ml ampicillin, 100 μg/ml spectinomycin, and 20 μg/ml chloramphenicol.

### Preculture Medium:

EcoK12 solution
Ultrapure water 1.0 kg
Citric acid monohydrate 40.0 g
Zinc sulfate heptahydrate 11.0 g
Diammonium iron sulfate hexahydrate 8.6 g
Manganese sulfate monohydrate 3.0 g
Copper sulfate pentahydrate 0.8 g
Cobalt sulfate heptahydrate 0.09 g

Sterilized by filtration using a filter with 0.2 μm pore size.

### Part 1

Ultrapure water 1.0 kg
Citric acid monohydrate 3.4 g
Magnesium sulfate heptahydrate 2.4 g
Calcium chloride dihydrate 0.1 g
EcoK12 solution 20 g
Sodium hydroxide solution 25% used to adjust pH to 6.6

### Part 2

Ultrapure water 500 g
Potassium dihydrogen phosphate 26.6 g
Diammonium hydrogen phosphate 8.0 g
Sodium hydroxide solution 25% used to adjust pH to 6.4

### Part 3

Ultrapure water 500 g
Glycerol 99% 36.0 g
Sodium gluconate 24.0 g
Phosphoric acid 20% used to adjust pH 6.6

All 3 parts were sterilized at 121° C. for 30 minutes.

### Vitamin Solution

Ultrapure water 100 g
Thiamine hydrochloride 1.0 g
Vitamin B₁₂ 0.5 g
   Sterilized by Filtration Using a Filter with 0.2 μm Pore Size

To make up the final preculture medium parts 1, 2, and 3 are combined and 2.0 ml of vitamin solution added. Furthermore, the medium was supplemented with 100 μg/ml ampicillin, 100 μg/ml spectinomycin, and 20 μg/ml chloramphenicol. Several transformants were scraped of the LB agar plate and used to inoculated 2×100 g of preculture media in 1 l baffled Erlenmeyer flasks. These precultures were incubated at 37° C. and 150 rpm. When an OD600 of 12 was reached the precultures were used in their entirety to inoculate the main culture.

### Main Culture Medium:

### Part 4

Ultrapure water 9.6 kg
Citric acid monohydrate 21.1 g
Potassium dihydrogen phosphate 173.6 g
Diammonium hydrogen phosphate 52.8 g
Magnesium sulfate heptahydrate 15.1 g
Calcium chloride dihydrate 0.7 g
EcoK12 solution 123 g
Sodium hydroxide solution 25% adjusted pH to 6.4
Pluriol P 2000 1 ml

Part 4 was sterilized at 125° C. for 45 min.

### Part 5

Ultrapure water 300 g
Thiamine hydrochloride 151 mg
Vitamin B12 30.2 mg
Ampicillin sodium salt 1000 mg
Spectinomycin hydrochloride 500 mg
Chloramphenicol 200 mg

Part 5 was sterilized by sterile filtration using a filter unit with a pore size of 0.1 μm

### Glycerol Solution

Ultrapure water 804 g
Citric acid monohydrate 29.1 g
Sodium sulfate 58.1 g
Diammonium iron sulfate hexahydrate 4.5 g
Glycerol 99% 3370 g

### Thiamine Solution

Ultrapure water 40 g
Thiamine hydrochloride 55 mg

### Antifoam Solution

Pluriol P 2000 350 g

### Base Solution

Ammonia water 25% 1500 ml

### Inductor Solution

Ultrapure water 150 g
Rhamnose monohydrate 100 g
IPTG 238 mg

Glycerol, and antifoam solution were sterilized at 121° C. for 30 min. Thiamine and inductor solution are sterilized by filtration using a filter with a pore size of 0.2 μm.

Parts 4 and 5 were combined in the sterilized fermentation vessel (Techfors, Infors HT) and inoculated with the preculture. The vessel was kept at a temperature of 37° C., a pressure of 0.2 bar, and at a pH of 6.6 by dosing with base solution over the course of fermentation. The pO2 level was kept at 20-40% by adjusting the stirrer speed (commonly 500 rpm) and aeration rate (commonly 6 l/min). Antifoam solution was added as needed. Glycerol and thiamine solutions were combined yielding the feed solution. After inoculation the feed solution was dosed at a rate of 10 g/h. After 7 h the dosing of the feed solution was switched to “stop and see” mode in which feed was activated at a rate of 10 g/h upon increase of pO2-level. After 14 h or 330 g of feed solution consumption the feed rate was increased to 80-100 g/h. Gene expression was induced at an oxygen transfer rate of 80 mmol/l/h or alternatively at an OD600 of 12 by addition of inductor solution. The fermentation was stopped 36 h post induction by lowering the temperature to 15° C. The cooled fermentation broth was drained from the fermenter and centrifuged at 4700 rpm and 10° C. to pellet the cells. The resulting supernatant was discarded, and cells resuspended in 3850 g of 50 mM potassium dihydrogen phosphate buffer at pH 7.0. The cell suspension was frozen at −80° C. before being lyophilized. In that regard, the lyophilizer was kept at −50° C. and a pressure of 0.25 mbar. Lyophilized cells were stored at 4° C.

### Production of Lyophilized Cell Free Extracts

Lyophilized cells were resuspended in ultrapure water at 100 g/l. The cell suspension was cooled on ice before cells were disrupted by three passages through a pressure homogenizer 40 (Panda Plus 2000, GEA) which was set to 800 bar. Pressures of the three passages were commonly between 1000 to 1400 bar. The resulting mixture was cleared from debris by centrifugation at 10000 rpm at 10° C. for 15 min. The resulting pellet was discarded and the concentration of protein in the supernatant analyzed by Bradford assay. The supernatant was frozen at −80° C. and subsequently lyophilized at −50° C. and a pressure of 0.25 mbar.

### Preparation of Starting Materials and Intermediate Products

### c) Chemical Synthesis of 5-([2-[butoxy(methyl)phosphoryl]ethyl]imidazolidine-2,4-dione (ex 3)

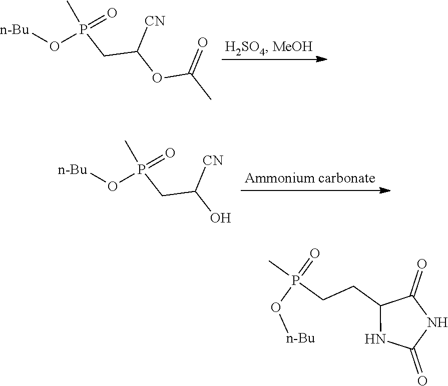

To a stirred solution of [2-[butoxy(methyl)phosphoryl]-1-cyano-ethyl] acetate (100 g, purity 90%, Cas 167004-78-6) in methanol (400 mL) was added concentrated sulfuric acid (1 g) and the reaction mixture was heated to 40° C. and stirred for 15 h at this temperature. The reaction mixture was allowed to cool to room temperature, then sodium methoxide in methanol (30%, 3.52 g) was added, followed by sodium sulfate (2 g) and stirred at room temperature for 30 min. The reaction mixture was filtered and the filtrate concentrated under reduced pressure (84.5 g).

To the crude butyl 3-cyano-3-hydroxypropyl(methyl)phosphinate (84.5 g, 366 mmol) was added a solution of diammonium carbonate (70.4 g, 732 mmol) in water (290 mL). The reaction mixture was heated to 70° C. for 4 h and then evaporated to dryness under reduced pressure. The residue was suspended in warm isopropanol (70° C.), the resulting suspension was filtered and the filter cake washed with isopropanol (2×10 mL). The filtrate was concentrated under reduced pressure and filtered through silica (elution with 1.5 L dichloromethane/methanol 9:1). The filtrate was concentrated under reduced pressure to yield 55.5 g of product and the resulting solid was recrystallized form isopropanol/diisopropylether (yield 34%). ¹H NMR (500 MHz, Deuterium Oxide) δ 4.42-4.37 (m, 1H), 4.08-4.00 (m, 2H), 2.19-1.77 (m, 4H), 1.70-1.64 (m, 2H), 1.61 (d, J=13.8 Hz, 3H), 1.46-1.34 (m, 2H), 0.92 (td, J=7.4, 0.8 Hz, 3H).

### d) Chemical Synthesis of 5-([2-[ethoxy(methyl)phosphoryl]ethyl]imidazolidine-2,4-dione from Racemic Glufosinate (Ex 4)

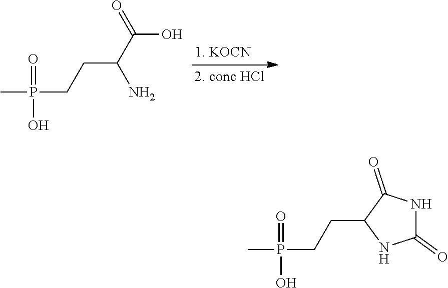

To a stirred solution of glufosinate ammonium (50% in water, 50 g, 126 mmol) under vacuum (200m bar) was added a solution of potassium cyanate (17 g, 202 mmol) in water (50 ml) at 50° C. over a period of 40 min. The reaction mixture was stirred at 50° C. under vacuum (200 mbar) for an additional 1.5 h and then allowed to cool to room temperature. After stirring at room temperature and ambient pressure for an additional 14 h, concentrated HCL (125 mL, 36%) was added and the reaction mixture was heated to reflux for 30 min. The reaction mixture was concentrated under reduced pressure. The residue was dissolved in water at 50° C. (50 mL) and filtered. The filtrate was subjected to ion exchange chromatography (Dowex-50 WX 8 200-400 (H), 500 mL) and the product eluted with water (1 L) yielding the product in virtually quantitative yield. ¹H NMR (500 MHz, Deuterium Oxide) δ 4.41-4.36 (m, 1H), 2.15-1.70 (m, 4H), 1.52 (d, J=13.9 Hz, 3H).

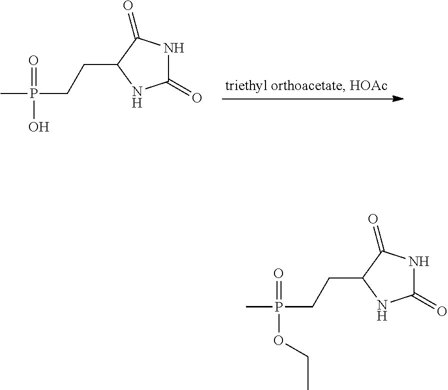

To a mixture of acetic acid (50 mL) and triethyl orthoacetate (75 mL, 409 mmol) was added 2-(2,5-dioxoimidazolidin-4-yl)ethyl-methyl-phosphinic acid (10 g, 48.5 mmol, synthesized as described above) at room temperature. The reaction mixture was heated to reflux (110° C., heating bath temperature) for 15 min. The reaction was then concentrated under reduced pressure and purified by column chromatography (dichloromethane/methanol 9:1) yielding ethyl 2-(2,5-dioxoimidazolidin-4-yl)ethyl(methyl)phosphinate (4.8 g, 42%).

¹H NMR (500 MHz, Deuterium Oxide) δ 4.41-4.36 (m, 1H), 4.13-4.03 (m, 2H), 2.19-1.74 (m, 4H), 1.60 (d, J=13.9 Hz, 3H), 1.35-1.28 (m, 3H).

### e) Synthesis of N-Carbamoyl Amino Acid (Under Chemical Conditions) (Ex 5)

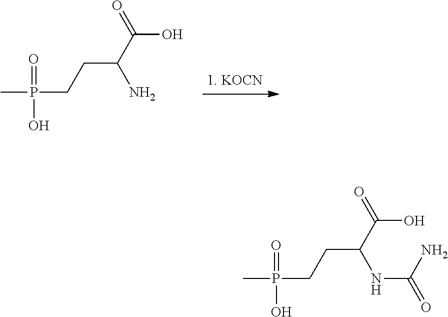

To a stirred solution of racemic glufosinate ammonium (50% in water, 39.6 g, 99.9 mmol) under vacuum (200m bar) was added a solution of potassium cyanate (11.8 g, 145 mmol) in water (30 ml) at 50° C. over a period of 30 min. The reaction mixture was stirred at 50° C. under vacuum (200 mbar) for an additional 1 h and then allowed to cool to room temperature. The reaction mixture was subjected to ion exchange chromatography (Dowex-50 WX 8 200-400 (H), 220 mL) and the product eluted with water (1 L). The eluted product was concentrated under reduced pressure yielding the carbamoylic acid product (7.9 g). The remaining carbamoylic acid was reisolated from the column as the potassium salt. 1H NMR (500 MHz, Deuterium Oxide) δ 4.31-4.25 (m, 1H), 2.19-1.81 (m, 4H), 1.52 (d, J=14.1 Hz, 3H). The D- and L-enantiomers were synthesized starting from commercially available D- and L-Glufosinate in an analogous fashion. Specific rotation for compound L-enantiomer [α]=+27.5° (c=1H₂O, measured as Potassium salt). HPLC-MS retention times using a Supelco Chirobiotic T2 (Eluent 40% water in Acetonitrile, 0.1% Formic acid). Temp: 20° C., flow rate 0.8 mL/min. Retention times of L-Carbamoyl amino acid (7.4 min); D-Carbamoyl amino acid (9.2 min).

### f) Chemical Synthesis of 5-([2-[ethoxy(methyl)phosphoryl]ethyl]imidazolidine-2,4-dione from N-carbamoyl Amino Acid (Ex 6)

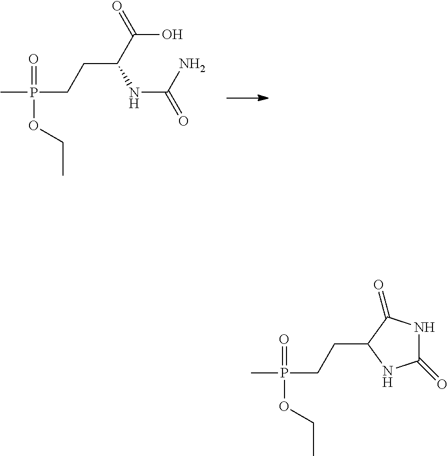 [SMILES: CCOP(C)(=O)CC[C@@H](NC(N)=O)C(=O)O ; CCOP(C)(=O)CCC1NC(=O)NC1=O]

(2R)-4-[ethoxy(methyl)phosphoryl]-2-ureido-butanoic acid (synthesized i.e. via Ex 11, or Ex 12) (164 mg) was dissolved in a solution of HCl in water (5%, 3 mL). The reaction mixture was shaken for 48 h at 40° C. NMR showed full conversion of the N-Carbamoyl amino acid to the D-hydantoin. The D-hydantoin can be readily racemized according to Example 14 (enzyme) or by treatment with aqueous ammonia at pH 8.5 (in an analogous fashion as described in example 15).

### Reaction Using a Chemical Carbamoyl Cleaving Step

### g) Enzymatic Synthesis of Butyl-Protected N-Carbamoyl Amino Acid (Ex 7)

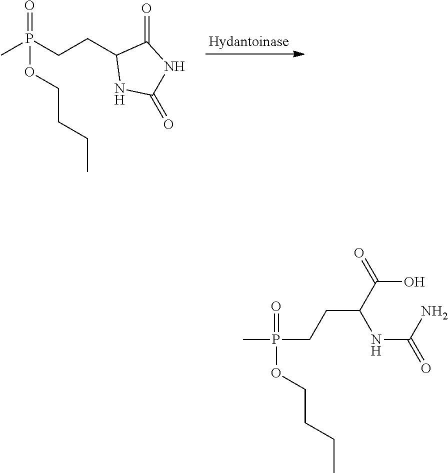 [SMILES: CCCCOP(C)(=O)CCC1NC(=O)NC1=O ; CCCCOP(C)(=O)CCC(NC(N)=O)C(=O)O]

To a solution of ([2-[butoxy(methyl)phosphoryl]ethyl]imidazolidine-2,4-dione (7.8 g, 29.7 mmol) in degassed aqueous potassium phosphate buffer (60 mL, 0.496 M, pH 8.0) was added KOH (3M in Water, 390 μL) to adjust the pH to 8.0. To the mixture was added potassium phosphate buffer (223 mL, 0.100 M, pH 8.0) and Hydantoinase enzyme (Uniprot ID:A0A159Z531_9RHOB, SEQ ID NO:1, cleared cell lysate, 16.5 mL, 8.1 mg/mL total protein concentration) and MnCl, solution (1 M in Water, 240 μL). The reaction mixture was stirred (250 rpm) at 37° C. for 24 h. The crude material was filtered to remove the cell lysate and the filter cake washed with water (60 mL). The filtrate was concentrated under reduced pressure, redissolved in THF/wet MeOH and filtered through silica (eluent pure methanol). The crude was then purified by reverse phase chromatography (water/acetonitrile 99:1 to 95:5 gradient) yielding 4-[butoxy(methyl)phosphoryl]-2-ureido-butanoic acid (2.05 g, 25%). 1H NMR (500 MHz, Deuterium Oxide) δ 4.11-4.07 (m, 1H), 4.06-4.00 (m, 2H), 2.09-1.80 (m, 4H), 1.71-1.63 (m, 2H), 1.59 (d, J=13.7 Hz, 3H), 1.46-1.34 (m, 2H), 0.92 (t, J=7.4 Hz, 3H).

### h) Enzymatic Synthesis of N-Carbamoyl Amino Acid (Ex 7a)

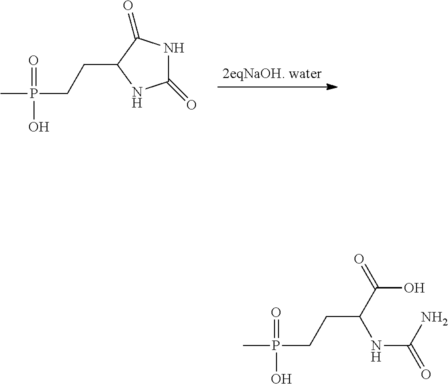

2-(2,5-dioxoimidazolidin-4-yl)ethyl-methyl-phosphinic acid (2.47 g) was dissolved in water (2 mL) and NaOH (50% in water, 1.91 g) was added. The reaction was diluted to a volume of 6 mL with water and shaken at 50° C. for 28 h. After this reaction time NMR revealed that 96% of the hydantoin was converted to the sodium salt of the N-carbamoyl Amino acid.

### i) Chemical Synthesis of Glufosinate (Ex 8)

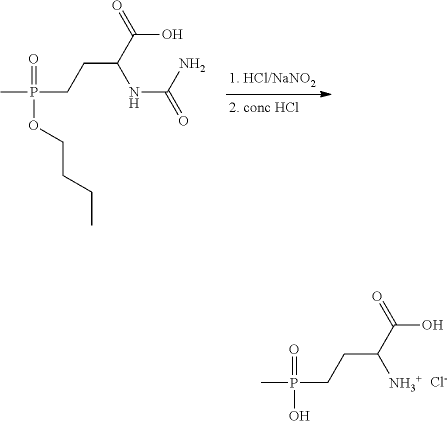 [SMILES: CCCCOP(C)(=O)CCC(NC(N)=O)C(=O)O ; CP(=O)(O)CCC([NH3+])C(=O)O ; [Cl-]]

4-[butoxy(methyl)phosphoryl]-2-ureido-butanoic acid (100 mg), synthesized using a Hydantoinase (Uniprot: A0A159Z531_9RHOB), was dissolved in aqueous HCl (3.5 M, 10 mL) and the stirred reaction mixture was cooled to 0° C. A solution of sodium nitrite (26 mg) in water (2 mL) was added and the reaction mixture was allowed to warm to room temperature. The reaction mixture was stirred at room temperature for an additional 2 hours. Then conc. HCl in water (36%, 7.5 mL) was added and the reaction mixture was heated to 100° C. and stirred at this temperature overnight. The reaction mixture was cooled to room temperature and extracted twice with methylene chloride (2×10 mL). The aqueous phase was concentrated under reduced pressure to obtain the hydrochloric acid salt of glufosinate. 1H NMR (500 MHz, Deuterium Oxide) δ 3.84-3.78 (m, 1H), 2.17-2.00 (m, 2H), 1.74-1.54 (m, 2H), 1.27 (d, J=13.5 Hz, 3H).

### j) Enzymatic Synthesis of Glufosinate (Ex 9)

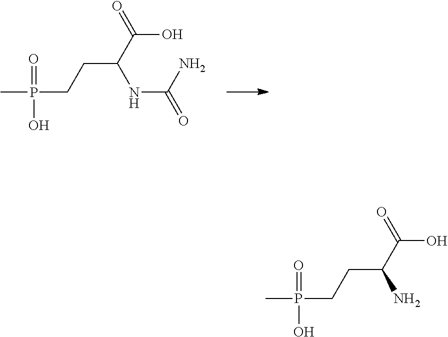

To a solution of 2-(carbamoylamino)-4-[hydroxy(methyl)phosphoryl]butanoic acid (0.6 g, 2.5 mmol) in degassed aqueous potassium phosphate buffer (5.4 mL, 0.496 M, pH 8.0) was added KOH (3M in Water) to adjust the pH to 8.0. To the reaction mixture (6.1 ml) was added potassium phosphate buffer (19.2 mL, 0.100 M, pH 8.0) and N-Carbamoyl amino acid hydrolase enzyme (Uniprot ID: A0A1Y4GC62_9BACT, SEQ ID NO:2, cleared cell lysate, 1.5 mL, 12.9 mg/mL total protein concentration, protein produced in shake flakes) and MnCl₂ solution (1 M in Water, 20 μL). The reaction mixture was stirred (250 rpm) at 37° C. for 24 h. NMR and HPLC analytics showed 31% conversion to Glufosinate. The enantiomeric ratio of glufosinate was analyzed by chiral HPLC. Chiral HPLC: >99%-L-Glufosinate/<1% D-Glufosinate; Analytical Method: Chirex (D)-Pencillamine 250x4.6 mm column from Phenomenex; isocratic elution 10 mM Copper (II) sulfate; UV detection at 245 nm).

### k) Enzymatic Synthesis of Glufosinate (Ex 10)

In parallel the reaction of Ex 9 was carried out with another N-Carbamoyl amino acid hydrolase enzyme under the same conditions (Uniprot ID: A0A6P2ISL4_BURL3, SEQ ID NO:3, cleared cell lysate, 1.5 mL, 10.2 mg/mL total protein concentration, protein produced in shake flakes) yielding also L-Glufosinate (21% conversion, Chiral HPLC: >99%-L-Glufosinate/<1% D-Glufosinate).

a) Enzymatic 1-Pot Synthesis of Ethyl-Glufosinate from Ethyl Ester of Hydantoin (Ex 11, SEQ ID NO: 1+4)

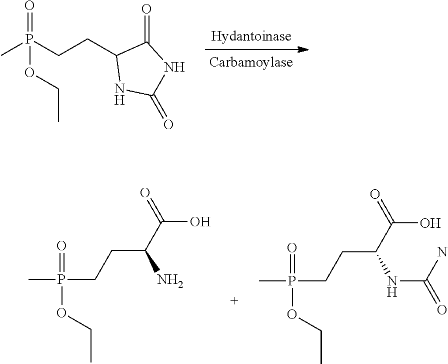 [SMILES: CCOP(C)(=O)CCC1NC(=O)NC1=O ; CCOP(C)(=O)CC[C@H](N)C(=O)O ; CCOP(C)(=O)CC[C@@H](NC(N)=O)C(=O)O]

To a solution of 5-[2-[ethoxy(methyl)phosphoryl]ethyl]imidazolidine-2,4-dione (5.6 g, 24 mmol) in Water (20 mL) was added Ammonia (25% in water) to adjust the pH to 8.5. To this mixture was added MnCl2solution (2M in Water, 1 mL) and Hydantoinase enzyme (Uniprot ID:A0A159Z531_9RHOB, SEQ ID NO:1, cleared cell lysate, 7.5 mL, 33 mg/mL total protein concentration) and N-Carbamoyl amino acid hydrolase enzyme (A0A535Y1H2_9CHLR, SEQ ID NO: 4, cleared cell lysate, 2.8 mL, 44.8 mg/mL total protein concentration). The reaction mixture was stirred at 37° C. for 72 h. During the 72 h reaction time the pH was kept at 8.5 with Ammonia (25% in water). After a total reaction time of 7 h, 27h, 30 h and 49 h N-Carbamoyl amino acid hydrolase enzyme (A0A535Y1H2_9CHLR, Seq ID: 4, 2.8 mL, cleared cell lysate) was added. NMR showed 36% conversion to the ethyl ester of L-Glufosinate after 24 h and 43% after 72 h. (Enantiomeric ratio by chiral HPLC L>99%, D>1%). After the reaction had finished, the crude reaction mixture was heated to 80° C. for 30 min and filtered to remove the cell lysate. The mixture of L-glufosinate ethyl ester and the ethyl ester of the N-carbamoyl amino acid was separated on a Dowex-50 WX 8 200-400 (H). The N-Carbamoyl amino acid was eluted with water and the ethyl ester of L-Glufosinate was eluted with ammonia (0.5 M in water) yielding the L-Glufosinate ethyl ester. Alternatively, the L-glufosinate ethyl ester could be separated by crystallization. The remaining carbamoyl amino acid can be recycled via Ex 6. The concentrations of the ethyl ester of L and D-Glufosinate were determined by HPLC-MS using a Supelco Chirobiotic T2 (Eluent 25% water in Acetonitrile, 0.1% Formic acid). Temp: 20° C., flow rate 1.0 mL/min. Retention times of Ethyl Ester of Glufosinate: L-configured Diastereoisomers (7.6+7.8 min); D-configured (8.5 and 11.5 min).

b) Enzymatic 1-Pot Synthesis of Ethyl-Glufosinate from Ethyl Ester of Hydantoin (Ex 12, SEQ ID NO: 1+3)

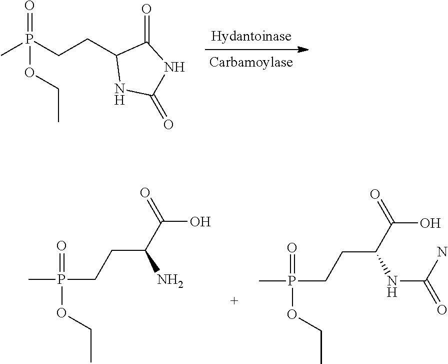 [SMILES: CCOP(C)(=O)CCC1NC(=O)NC1=O ; CCOP(C)(=O)CC[C@H](N)C(=O)O ; CCOP(C)(=O)CC[C@@H](NC(N)=O)C(=O)O]

To a solution of 5-[2-[ethoxy(methyl)phosphoryl]ethyl]imidazolidine-2,4-dione (5.6 g, 24 mmol) in Water (20 mL) was added Ammonia (25% in water) to adjust the pH to 8.5. To this mixture was added MnCl₂solution (2M in Water, 1 mL) and Hydantoinase enzyme (Uniprot ID:A0A159Z531_9RHOB, SEQ ID NO:1, cleared cell lysate, 7.5 mL, 33 mg/mL total protein concentration) and N-Carbamoyl amino acid hydrolase enzyme (A0A6P2|SL4_BURL3, SEQ ID NO:3, cleared cell lysate, 2.8 mL, 44 mg/mL total protein concentration). The reaction mixture was stirred at 37° C. for 48 h. During the 48 h reaction time the pH was kept at 8.5 with Ammonia (25% in water). After a total reaction time of 7 h, 27h and 30 h N-Carbamoyl amino acid hydrolase enzyme (SEQ ID NO:3, 2.8 mL, cleared cell lysate) was added. NMR showed 24% conversion to the ethyl ester of L-Glufosinate (enantiomeric ratio L:D>99:1). The concentrations of the ethyl ester of L and D-Glufosinate were determined by HPLC-MS using a Supelco Chirobiotic T2 (Eluent 25% water in Acetonitrile, 0.1% Formic acid). Temp: 20° C., flow rate 1.0 mL/min. Retention times of Ethyl Ester of Glufosinate: L-configured Diastereoisomers (7.6+7.8 min) D-configured (8.5 and 11.5 min).

### c) Enzymatic Synthesis of N-Carbamoyl Amino Acid (Ex 13, SEQ ID NO:1)

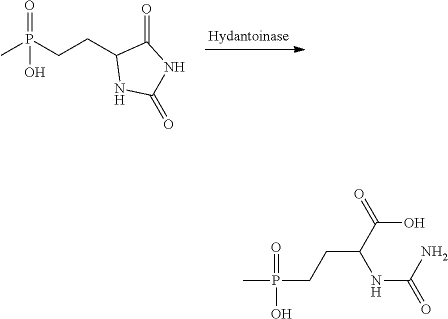

2-(2,5-dioxoimidazolidin-4-yl)ethyl-methyl-phosphinic acid (25 g) was dissolved with heating in aqueous ammonia solution (53 mL, 10 M). The reaction was cooled to 37° C. and the pH adjusted to 8.7 using ammonia. MnCl₂solution (2M in Water, 2.5 mL) and Hydantoinase enzyme (Uniprot ID:A0A159Z531_9RHOB, SEQ ID NO:1, lyophilized cell free extract, 1.28 g) were added and the pH was adjusted to 8.7 using aqueous ammonia solution. The reaction mixture was stirred at 37° C. for 72 h and the pH was continuously adjusted to 8.7 using 10 M Ammonia solution. After 24 h HPLC showed 95% conversion of the Hydantoin to the Carbamoylic acid. HPLC Conditions: The conversion of hydantoin to carbamoylic acid was determined by HPLC-MS using a Luna C8 150×, 3.0 mm column (water+0.1% formic acid). Temp: 40° C., flow rate 0.5 mL/min. Retention times: Hydantoin 3.4 min, N-Carbamoyl amino acid 2.5 min.

### d) Racemization of Hydantoin (Ethyl-Ester) Using a Racemase at pH 7.0 (Ex 14, Racemase A0A6V7ACK5 RHIRD, Seq ID: 5)

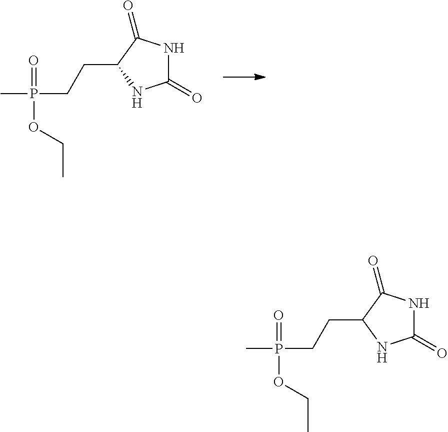

(5R)-5-[2-[ethoxy(methyl)phosphoryl]ethyl]imidazolidine-2,4-dione (37.5 mg, ratio between D-configured hydantoin and L-configured 95/5) was dissolved in water (75 μL) and the pH was adjusted with Ammonia (10M in water) to 7.0. To this mixture was added Hydantoin Racemase (Seq ID:5; A0A6V7ACK5_RHIRD, 20.2 mg/ml, 150 μL, cleared cell lysate) followed by 1.6 μL MnCl₂ (2 M in water). The reaction mixture was shaken at 37° C. for 24 h. After a total reaction time of 4 h the ratio D/L-Hydantoin had changed from 95/5 to 53/47. After 20 h racemization of the hydantoin was almost complete (Ratio 51/49). The concentration of L and D-Hydantoin were determined by HPLC-MS using a Supelco Chirobiotic T2 (Eluent 25% water in Acetonitrile, 0.1% Formic acid). Temp: 20° C., flow rate 0.8 mL/min. Retention times of Ethyl Ester of Glufosinate: D-configured Diastereoisomers (5.8+6.1 min); L-configured (7.2 and 10.5 min).

### e) Racemization of Hydantoin at pH 8.5 (Ex 15)

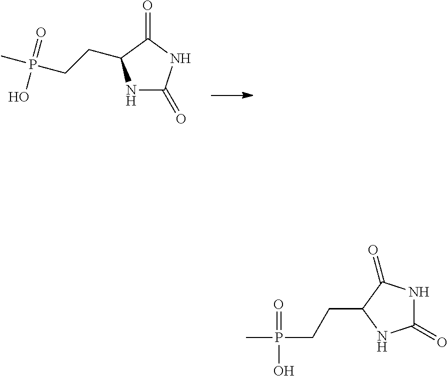

2-[(4S)-2,5-dioxoimidazolidin-4-yl]ethyl-methyl-phosphinic acid (206 mg, enantiomeric ratio L:D 92:8, measured by chiral HPLC) was dissolved in 900 μL water and the pH was adjusted to pH 8.5 by using 50 μL Ammonia (10 M in water). To the reaction mixture was added 10 μL of a 2 M MnCl₂ solution, followed by 30 μL of water. The reaction mixture was shaken at 37° C. for 24 h hours. After a total reaction time of 3h the enantiomeric ratio was L:D 53:47, and after a total reaction time of 24 h it was 50:50. This shows that the hydantoin readily racemizes under concentrated basic conditions in aqueous ammonia. The concentrations of L and D-Hydantoin were determined by HPLC-MS using a Supelco Chirobiotic T2 (Eluent 40% water in Acetonitrile, 0.1% Formic acid). Temp: 20° C., flow rate 0.8 mL/min. Retention times of L-Hydantoin (11.3 min min); D-Hydantoin (6.6 min).

### f) Racemization of Hydantoin Using a Racemase at pH 7.8 (Ex 16, Racemase A0A2T6KHH4_9RHOB, Seq ID: 6)

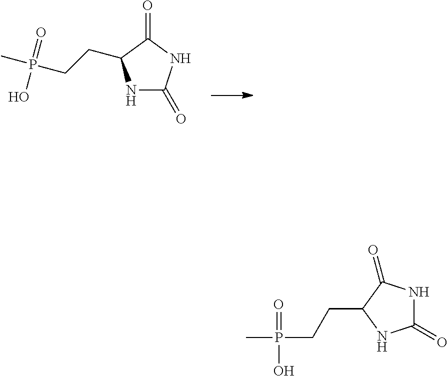

2-[(4S)-2,5-dioxoimidazolidin-4-yl]ethyl-methyl-phosphinic acid (206 mg, enantiomeric ratio L:D 92:8, measured by chiral HPLC) was dissolved in 500 μL water and the pH was adjusted to pH 7.8 by using Ammonia (10 M in water). To this mixture was added Hydantoin Racemase (A0A2T6KHH4_9RHOB, Seq ID: 6, 100 μL, cell free extract, 26.1 mg/mL total protein concentration) followed by 10 μL MnCl₂ (2 M in water). The reaction volume was adjusted to 1 mL and the pH adjusted to 7.8 by Ammonia (10 M in water). The reaction mixture was shaken at 37° C. for 24 h hours. After a total reaction time of 2h the enantiomeric ratio was L:D 50:50.

g) Enzymatic Screening for novel biocatalysts (Ex 17)

E. coli TG10 containing the plasmids pAgro4 and pHSG575 were transformed with pDHE plasmid encoding the protein of interest. A resulting single clone was used to inoculate 1 ml of preculture medium (see Fermentative whole-cell biocatalyst production, EX2) supplemented with 1 mM MnCl2, 100 μg/ml ampicillin, 100 μg/ml spectinomycin, and 20 μg/ml chloramphenicol in a well of a 48-well flower shaped microtiter plate (m2plabs). Cultures were incubated at 37° C. and 1000 rpm overnight. For the main culture preculture medium (see Fermentative whole-cell biocatalyst production, EX2) was supplemented with 1 mM MnCl2, 100 μg/ml ampicillin, 100 μg/ml spectinomycin, 20 μg/ml chloramphenicol, 1 mM IPTG, and 1% rhamnose. 1 ml of the resulting medium was dispensed in a well of a 48-well flower shaped microtiter plate (m2plabs) and inoculated with 10 μl of preculture. The main culture was incubated overnight at 37° C. at 1000 rpm. Subsequently, cells were pelleted by centrifugation at 3750×g, at 4° C. for 15 min and the supernatant discarded. For screenings using whole cells, cell pellets were resuspended in 500 μl 50 mM HEPES buffer at pH 8.4 supplemented with 1 mM MnCl2. In case cleared cell lysates were used, cell pellets were resuspended in 500 μl 50 mM HEPES buffer at pH 8.4 supplemented with 1 mM MnCl2, 1 mg/ml lysozyme, 0.3 mg/ml polymyxin b sulfate, 0.01 mg/ml DNase, 0.01 mg/ml RNase, and the suspension incubated at room temperature and 1000 rpm for one hour. Resulting cell lysates were cleared from debris by centrifugation 3750×g, at 4° C. for 20 min. 50 μl of the cleared cell lysate or of the whole cell suspension were used in a 200 μl reaction containing 10 mM of the relevant substrate, and 1 mM MnCl2 in 100 mM HEPES at pH 8.4. Reactions were run overnight at 37° C. before being quenched with TFA at final concentration of 5%. Precipitates were removed by centrifugation and the supernatant subjected to analyte quantification using HPLC coupled to a mass spectrometry detector. HPLC-MS employing a Luna C8 150×, 3.0 mm column (water+0.1% formic acid). Temp: 40° C., flow rate 0.5 mL/min was used for the detection of N-Carbamoylic acid, Hydantoin and Glufosinate itself, whereas the molecules containing the butyl ester were separated on a Kinetex C18 100x2.1 mm column (Flow rate 0.5 mL/min, 20% Acetonitrile in water with 0.1% formic acid).

1) Hydantoinases were screened using whole cells and with 10 mM of racemic 5-([2-[ethoxy(methyl)phosphoryl]ethyl]imidazolidine-2,4-dione (Glufosinate hydantoin, Synthesis Ex3) or 5-([2-[butoxy(methyl)phosphoryl]ethyl]imidazolidine-2,4-dione (butyl ester of glufosinate hydantoin, Synthesis EX4) as substrate. Formation of the respective N-carbamoyl amino acid from the hydantoinase was monitored. Hydantoinases Q45515, Q44184, A0A1C4QIY5_9ACTN, A0A0K2UMP4_LEPSM, *WP_046170519.1, and E1R8C9_SEDSS showed 2-(carbamoylamino)-4-[hydroxy(methyl)phosphoryl]butanoic acid (N-Carbamoylic acid of glufosinate) yields of >0.1%. Hydantoinases 069809, Q846U5_9BACL, P81006, Q84FR6_9MICC, Q56S49_9BACI, A1E351_9BACI, Q28SA7, Q45515, A0A399DRQ3_9DEIN, Q55DL0, F7X5M8_SINMM, Q91676, Q44184, B5L363, P42084, P25995, Q3Z354, B1XEG2, Q9F465_PAEAU, A0A161KD37_9CHLR, A0A1J4XHR4_9BACT, A0A1C4QIY5_9ACTN, A0A0K2UMP4_LEPSM, A0A159Z531_9RHOB, E1R8C9_SEDSS, A0A1F9QT17_9BACT, A0A0D81VV8_9FIRM, A0A0B5QKE4_CLOBE, A0A0N1GBZ8_9ACTN, A0A174ADZ3_9FIRM, U7V9Q6_9FUSO, A0A0J1FA14_9FIRM, PHYDA_ECOK1, A0A0S8H576_9BACT, A0A1J4J4Y8_9EUKA, A0A0D5NFS5_9BACL, A0A0D5NNJ7_9BACL, A0A1H2AV66_9BACL, A0A0Q4RXY0_9BACL, A0A0Q7SB75_9BACL, A0A100VRN2_PAEAM, W4BDJ0_9BACL, A0A1J5E082_9DELT, A0A1H5ZFN3_9BACT, A0A1F8NMM2_9CHLR, A0A1F8SDV1_9CHLR, A0A1H1PLX0_9BACT, A0A0Q518X4_9DEIO, *WP_046170519.1, *WP_023514195.1, *WP_023516147.1, and *ANZ15483.1 showed 4-[butoxy(methyl)phosphoryl]-2-ureido-butanoic acid (N-Carbamoyl acid of Glufosinate-Butylester) yields of >0.1%.

2) Carbamoylases were screened using cleared cell lysates and 10 mM 2-(carbamoylamino)-4-[hydroxy(methyl)phosphoryl]butanoic acid (N-Carbamoylic acid of glufosinate) or 4-[butoxy(methyl)phosphoryl]-2-ureido-butanoic acid (N-Carbamoyl acid of Glufosinate-Butylester) as substrate. Formation of Glufosinate or the butyl ester of glufosinate was monitored. Carbamoylases A0A3E0C996_9BURK, A0A535Y1H2_9CHLR, A0A6P21SL4_BURL3, and A0A1Y4GC62_9BACT showed Glufosinate yields of >0.1%. Carbamoylases A0A0K9YX84_9BACL, E3HUL6_ACHXA, Q9F464, A0A4D7Q548_GEOKU, Q9F464, A0A2S9D976_9MICC, A0A3E0C996_9BURK, A0A535Y1H2_9CHLR, A0A6P21SL4_BURL3, and A0A1Y4GC62_9BACT showed butyl ester of Glufosinate yields of >0.1%

### h) Enzymatic Cascade Reaction on Small Scale (Ex 18)

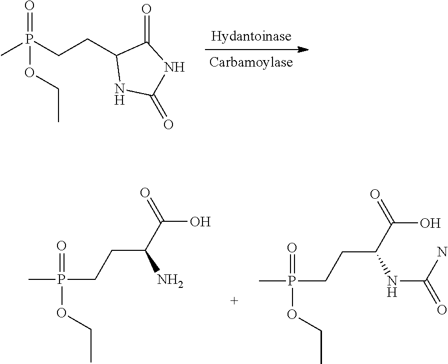 [SMILES: CCOP(C)(=O)CCC1NC(=O)NC1=O ; CCOP(C)(=O)CC[C@H](N)C(=O)O ; CCOP(C)(=O)CC[C@@H](NC(N)=O)C(=O)O]

Lyophilized cell free extracts were solved in 1 M HEPES buffer at pH 8.4. Reactions were set up at 400 μl scale in 1 M HEPES buffer at pH 8.4 containing 75 mM MnCl₂, and 100 mM racemic 5-[2-[ethoxy(methyl)phosphoryl]ethyl]imidazolidine-2,4-dione. Reactions were initiated by the addition of hydantoinase Q44184 (SEQ ID 7) and carbamoylase A0A535Y1H2_9CHLR (SEQ ID 4) at a final concentration of 19 mg/ml and 7.3 mg/ml, respectively. Subsequently, reactions were incubated at 37° C. for 24 hours before being stopped by heating to 95° C. for 5 min. Precipitates were removed by centrifugation, the supernatant diluted 100-fold, and subjected to analyte quantification using chiral HPLC coupled to a mass spectrometry detector. The reaction yield for the ethyl ester of glufosinate was 22.3% with an enantiomeric ratio of L>99%, D>1%.

i) Enzymatic 1-Pot Synthesis of Glufosinate from N-Carbamoyl Amino Acid (Ex 19, SEQ ID NO:1+3)

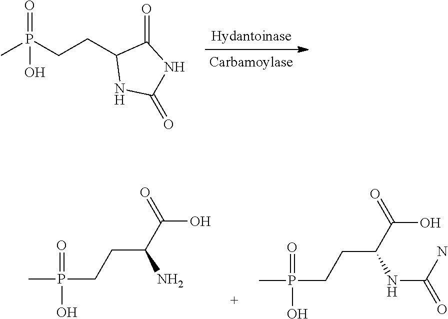

To a solution of 2-(2,5-dioxoimidazolidin-4-yl)ethyl-methyl-phosphinic acid (7.3 g) in 30 mL of aq. Ammonia (2M) was added aq. Ammonia (25% in water) to adjust the pH to 8.0. To this mixture was added MnCl₂solution (2M in Water, 1 mL) and Hydantoinase enzyme (Uniprot ID:A0A159Z531_9RHOB, SEQ ID NO:1, lyophilized cleared cell lysate, 1.2 g) and N-Carbamoyl amino acid hydrolase enzyme (Uniprot ID: A0A6P21SL4_BURL3, cleared cell lysate, 2.8 mL, 44 mg/mL total protein concentration). The reaction mixture was stirred at 37° C. for 44 h. After a total reaction time of 4 h N-Carbamoyl amino acid hydrolase enzyme (A0A6P21SL4_BURL3, 2.8 mL) was added, after a total reaction time of 23 h it was added again (A0A6P21SL4_BURL3, 11.2 mL). After 44 h 83% of the hydantoin had converted to the N-Carbamoyl amino acid and 10% to Glufosinate as measured by NMR. Chiral HPLC Analytics showed an enantiomeric ratio of L-Gufosinate: D-Glufosinate 92:8. The ratio between L- and D-Glufosinate was determined by H PLC-MS using a Supelco Chirobiotic T2 (Eluent 40% water in Acetonitrile, 0.1% Formic acid). Temp: 20° C., flow rate 0.8 mL/min. Retention times of L-Glufosinate (6.8 min min): D-Glufosinate (7.4 min).The remaining carbamoyl amino acid can be recycled via Ex 6.

SEQ ID NO:1 (from Defluviimonas alba)

MTLIVTNGRVVSPEGVALRDVVVEGETIAAVLPAGEAVKACPGAEVIDAT
GRIVIPGGVDPHVHLLVGFMGQRSVYDFASGGIAALRGGTTAIVDFALQR
RGGSMLKGLAHRRKQADANVTLDYGLHLIVTDVTADTLAELPALRAAGVT
TLKVYTVYEEDGLKVEDGALFALMQGAARHGLSVVLHAENAGIVERLRAE
AVARGDTHPRHHALTRPPIVEIEAVSRAIAFSRATGCGVHILHLVAADAI
ALVAAARAEGLPVTAETCSHYLALTDEALERPNGHEFILSPPLRDKANQD
RLWKGLETAALSLVASDEVSYSAAAKAMGLPSFATVANGITGIEARLPLL
YTLGVDQGRIGLQRFVKLFSTWPAEIFGFAGKGRIAPGFDADLVLIDPDG
RRVISTDSDYGDIGYTPYAGMELTGFATETIYRGRLVVRDGVFLGTEGQG
RFIERVAPRRPAP

SEQ ID NO:2 (from Cloacibacillus sp. An23)

MNCVNDILRSIGKAGRNEDGSYTRACYSAEYFAAVDITEKLMREYGMETS
RDAAGNLHGVLPGTEPGLKSIIIGSHLDTVPEGGLFDGAYGVAGGLEVVR
RLKEEGRRPRHTIELYGFNAEESSPLGGTFGSRAVTGLVSPEQPGLAEAL
KSYGHTVEEIMGCRRDFSDAKCYLELHIEQGDYLFSEGQKIGVVSGIVGV
IRYKVTALGHSNHAGTTMMKNRRDAMVAMARLITEADRRCRAIDDRLVLT
VGTIKCWPGSENVIPGKVECSFEMRHMDKAKTDELIREIREIAENIATVE
FEIVNMIDKGAVSCDAHLMDVICEAAEEAGESHVVMPSGAGHDANPMAHR
VPIGMIFVPSKDGMSHCPEEWTDSEETAAGAEVLYRTVLALDAED

SEQ ID N0:3 (from Burkholderia lata)

MNPTDFPFPPLNAERLNARVEQLARFTRPDVPWTRRAFSPLFTEARAWLA
AQFAEAGLAVSMDAGGNLIGRREGSGRCTKPLVTGSHCDTVVGGGRFDGI
IGVLAGIEVAHTLNEQGIVLDHPFEVIDFLSEEPSDYGISCVGSRALSGV
LDAGMLRATNAEGETLAEALRRIGGNPDALREPLRAPGSTAAFVELHIEQ
GPVLETRGLPIGVVTNIVGIRRVLITVTGQPDHAGTTPMDIRRDALVGAA
HLIEAAHARASALSGNPHYVVATIGRIAMTPNVPNAVPGQVELMLEVRSD
SDAVLDAFPEALLAGAAARLDALRLSARAEHVSRARPTDCQPLVMDAVEQ
AATQLGYPSMRLPSGAGHDAVYVAPTGPIGMIFIPCLGGRSHCPEEWIEP
QQLLDGTRVLYQTLVALDRSLAGAA

SEQ ID NO:4 (from Chloroflexi bacterium)

MTDAARLERRIHELAQIGRTDDPAREIYATAVSRLGLSAEEQRARDLVTS
WCAPHGATARRDPAANLYLRFPGADPHAPVVLVGSHLDSVPMGGRFDGAL
GVCCAVEAVVSLLESGARFARPVEVVGWADEEGARFGYGLFGSAAAFGRL
RVDPERVRDKGGTSIAEALRALGESGDLAGAMRDPKGIRAYLELHIEQGP
RLERAGAPLGVVSDIVGIFHGLVMVRGEQNHAGATVMGERHDALVAASHM
IIALERIASSVPDAVATVGEITVKPGAKNVIPGECTFSLDIRAPKQESID
LVLERFKAEANEIFRKSLREWGLRPLQSVAVTPLDEDLRDLLWKSAMSVG
VNAPTLVSGAGHDAQNPSLAGVPTGMIFVRSTGGSHTPTEFAATADAALG
AKALEIAIRELATA

SEQ ID NO: 5 (from Rhizobium radiobacter (Agrobacterium tumefaciens))

MHIRLINPNSTASMTAQALDSALRVKQKDTHVSAANPVDTPVSIEGQADE
AMAVPGLLAEIRKGEGHGVDAYVIACFDDPGLHAAREVARGPVIGICQAA
VQVAMTISRRFSIITTLPRSIPIIEDLVEDYGAQRYCRKVRAIDLPVLGL
EEDPEVAEALLRREIEAAKREDAAEAIILGCAGMSSLCDRLRDATGVPVI
DGVTAAIKLAEALVGAGYTTSKVNAYDYPRVKGPALVACA

SEQ ID N0:6 (from Yoonia sediminilitoris)

MSALIIINPNSSQTVTDGIDAAVAPLRSFGTPIRCLTLAEGPPGIESQKQ
ADLTVAPMLKLAAEQADAAGYVIACFGDPGLHALRDQTHLPVVGIQEAAV
MTALTLGQRFGVIAIMPGSIPRHLRAFGAMSVLDRLAGDRALGLGVADLA
DPDRSLAAMIATGKRLRDEDGAHVLIMGCAGMAHYRPTLETETGLPVVEP
CQAATAMVLGHIALGQSHRRDQN

SEQ ID NO:7 (from Rhizobium radiobacter) (Agrobacterium tumefaciens) (Agrobacterium 15 radiobacter)

MDIIIKNGTIVTADGISPADLGIKDGKIAQIGGTFGPAGRTIDASGRYVF
PGGIDVHTHVETVSFNTQSADTFATATVAAACGGTTTIVDFCQQDRGHSL
REAVAKWDGMAGGKSAIDYGYHIIVLDPTDSVIEELEVLPDLGITSFKVF
MAYRGMNMIDDVTLLRTLDKAAKTGSLVMVHAENGDAADYLRDKFVADGK
TAPIYHALSRPPRVEAEATARALALAEIVNAPIYIVHLTCEESFDELMRA
KARGVHALAETCTQYLYLTKDDLERPDFEGAKYVFTPPPRTKKDQEILWN
ALRNGVLETVSSDHCSWLFEGHKDRGRNDFRAIPNGAPGVEERLMMVYQG
VNEGRISLTQFVELVATRPAKVFGMFPEKGTVAVGSDADIVLWDPEAEMV
IEQSAMHNAMDYSSYEGHKIKGVPKTVLLRGKVIVDEGTYVGAPTDGQFR
KRRKYKQ

1. A method of manufacturing glufosinate, its alkyl ester or the salts thereof having the formula (3) 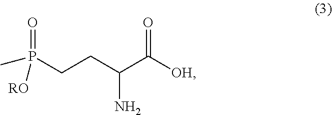
wherein R is H or C1-C8alkyl, comprising the step of enzymatically cleaving off the carbamoyl moiety of a N-carbamoyl amino acid having the formula (2) 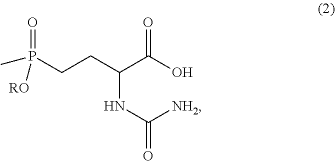
wherein R is H or C1-C8alkyl.

2. The method according to claim 1, wherein the cleaving step provides glufosinate, its alkyl ester or the salts thereof having the formula (3) 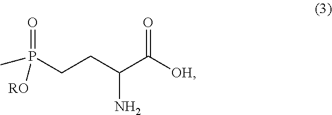
wherein R is H or C1-C8alkyl.

3. The method according to claim 1, wherein the cleaving step provides glufosinate, its alkyl ester or the salts thereof having the formula (3) in form of a racemic mixture or in form of an enantiomeric excess of L-glufosinate, its alkyl ester or the salts thereof having the formula (3a) 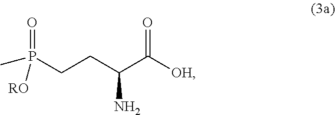
wherein R is H or C1-C8alkyl.

4. The method according to claim 3, wherein at least 50% of the N-carbamoyl amino acid having the formula (2) is converted to L-glufosinate, its alkyl ester or the salts thereof having the formula (3a), wherein formula (3a) is as defined in claim 3.

5. The method according to claim 1, wherein the cleaving is performed by an N-Carbamoyl amino acid hydrolase enzyme.

6. The method according to claim 1, wherein the cleaving step is performed by an N-Carbamoyl amino acid hydrolase enzyme selected from the group of enzymes identified by their Uniprot ID consisting of A0A0K9YX84_9BACL and variants thereof, E3HUL6_ACHXA and variants thereof, Q9F464 and variants thereof, A0A4D7Q548_GEOKU and variants thereof, Q9F464 and variants thereof, A0A2S9D976_9MICC and variants thereof, A0A3E0C996_9BURK and variants thereof, A0A535Y1H2_9CHLR and variants thereof, A0A6P2ISL4_BURL3 (SEQ ID NO:3) and variants thereof, A0A1Y4GC62_9BACT (SEQ ID NO:2) and variants thereof, wherein variants are defined as polypeptide sequences with at least 80% sequence identity to the respective polypeptide sequence.

7. The method according to claim 1, wherein R in formulae (2) and (3) is H or C1-C6alkyl.

8. The method according to claim 1, wherein the cleaving step is performed at a pH of 6 to 11 and/or
at a temperature of 20 to 50° C.

9. The method according to claim 1, wherein R in formulae (2) and (3) is C1-C8alkyl, and the method further comprises the step of
c) deprotecting under acidic conditions.

10. The method according to claim 1, wherein the method further comprises the addition of an N-Carbamoyl amino acid racemase enzyme.

11. The method according to claim 1, wherein the N-carbamoyl amino acid having the formula (2) is provided by a preceding hydrolysing step comprising hydrolysing a hydantoin having the formula (1) 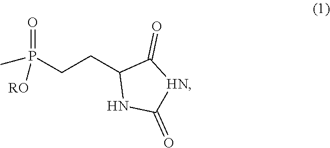
wherein R is H or C1-C8alkyl, to form the N-carbamoyl amino acid having the formula (2).

12. The method according to claim 1, wherein the N-carbamoyl amino acid having the formula (2) is provided by a preceding hydrolysing step, wherein hydrolysing the hydantoin having the formula (1) is performed by a Hydantoinase enzyme.

13. The method according to claim 11, wherein the hydrolysing step and the cleaving step are performed in a single container.

14. A composition comprising a N-carbamoyl amino acid having the formula (2a) 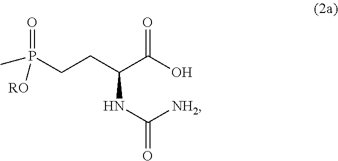
wherein R is H or C1-C8alkyl, and L-glufosinate or the salts thereof.

15. A method for selectively controlling weeds in an area containing a crop of planted seeds or crops that are resistant to glufosinate, comprising:
applying an effective amount of a composition comprising L-glufosinate or the salts thereof at an enantiomeric proportion of at least 80% over D-glufosinate or the salts thereof and more than 0.01 wt.-% to less than 10 wt.-%, based on the total amount of the composition, of a N-carbamoyl amino acid having the formula (2) 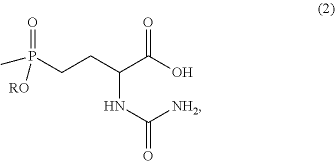
wherein R is H or C1-C8alkyl, to the area.
